(19) United States
(12) Patent Application Publication
(10) Pub. No.: US 2025/0266172 A1
(43) Pub. Date: Aug. 21, 2025

(54) COMPUTER INTERVENTION RESPONSE EXTRACTION SYSTEM

(71) Applicant: Flatiron Health, Inc., New York, NY (US)

(72) Inventors: Konstantin Krismer, New York, NY (US); Prakrit Baruah, New York, NY (US); Edward Liu, Brooklyn, NY (US); Samuel Wilkinson, New York, NY (US); Auriane Blarre, New York, NY (US)

(21) Appl. No.: 19/001,198

(22) Filed: Dec. 24, 2024

Related U.S. Application Data
(60) Provisional application No. 63/556,341, filed on Feb. 21, 2024.

(51) Int. Cl. G16H 50/70; G06F 40/279; G16H 10/60

(52) U.S. Cl. CPC G16H 50/70; G16H 10/60; G06F 40/279

(57) ABSTRACT

Described herein are techniques of automatically extracting intervention responses of a subject from data associated with the subject. The system automatically determines time points (e.g., dates) indicating periods in which intervention responses were determined for subjects, and then uses the time points to identify datasets from which to extract intervention responses. The system extracts intervention responses of the subject from the identified datasets.

### RELATED APPLICATIONS

This application claims priority under 35 U.S.C. 119 (e) to U.S. Provisional Application Ser. No. 63/556,341 entitled “COMPUTER INTERVENTION RESPONSE EXTRACTION SYSTEM,” filed on Feb. 21, 2024, which application is incorporated herein by reference in its entirety.

### FIELD

Described herein are techniques for computer-based extraction of subjects' responses to interventions from datasets associated with the subjects.

### BACKGROUND

Clinical record data may be collected about a subject (e.g., a patient) over time and stored in an electronic health record (EHR). The EHR may be used by a healthcare system to collect and store medical information about multiple different subjects. The clinical record data may include various types of information about the subject such as information about medical history, diagnoses of condition(s), intervention (e.g., medication), intervention response, laboratory data, and/or other types of information.

### SUMMARY

Real-world data (RWD) is clinical record data associated with subjects (e.g., patients). The data may include information about a subject's health (e.g., diagnosed illness, stage of illness, age, weight, height, allergies, date of birth, and/or other information) and delivery of healthcare (e.g., prescribed drugs, medical test history, medical image data, and/or other information). RWD can come from various sources including electronic health records (EHRs), insurance claims, monitoring devices (e.g., wearable devices, at home monitoring devices), mobile devices, and/or other sources.

RWD allows researchers to go beyond data gathered through a controlled trial. Data from controlled trials is limited to the characteristics of a cohort examined in a trial. Moreover, obtaining data through controlled trials requires significant investment and time. By contrast, RWD can be collected from any number of groups. Insights gained from the data can be valuable for use in medical diagnosis, medical treatment, development of treatments (e.g., procedures, drugs, and/or other types of treatments), life science research, and/or other purposes.

An important data point in RWD about a subject is the subject's response to an intervention (e.g., chemotherapy) for a condition (e.g., a tumor present in the subject). This data point is a key measure of intervention efficacy and in regulatory approval of an intervention (e.g., as part of a clinical trial). Described herein are techniques for automatically extracting intervention responses of a subject from data associated with the subject. The system automatically determines time points (e.g., dates indicating periods in which intervention responses were determined for subjects) and then uses the time points to identify datasets from which to extract intervention responses. The system extracts the intervention responses of the subject from the identified datasets.

Some embodiments provide a computer system for automatically extracting intervention responses from data associated with subjects, the computer system comprising: at least one processor; and a plurality of modules executed by the at least one processor, the plurality of modules comprising a time point extraction module, a response data identification module, and a response extraction module, wherein: the time point extraction module is configured to: access imaging related data associated with a subject; process the imaging related data using a first trained machine learning (ML) model to obtain a plurality of time points (e.g., indicating respective predicted time periods of intervention response determination for the subject), the processing comprising: generate a plurality of sets of features using data from the image related data; and process the plurality of sets of features using the first trained ML model to obtain the plurality of time points; the response data identification module is configured to: generate, using the data associated with the subject, a dataset collection for each of at least some of the plurality of time points to obtain a plurality of dataset collections, the generating comprising: identify, in the data associated with the subject, one or more datasets generated after the time point; and include the one or more datasets in the dataset collection; and the response extraction module is configured to: process the plurality of dataset collections using a second trained ML model to obtain a plurality of intervention responses, the processing comprising, for each of at least some of the plurality of dataset collections: generate a set of features using data from the dataset collection; and process the set of features using the second trained ML model to obtain an intervention response of the plurality of intervention responses.

Some embodiments provide a method for automatically extracting intervention responses from data associated with subjects. The method comprises: using at least one processor to perform: accessing imaging related data associated with a subject; processing the imaging related data using a first trained machine learning (ML) model to obtain a plurality of time points, the processing comprising: generating a plurality of sets of features using data from the image related data; and processing the plurality of sets of features using the first trained ML model to obtain the plurality of time points; identifying, from the data associated with the subject, a first collection of one or more datasets generated after a first one of the plurality of time points; generating, using the data associated with the subject, a dataset collection for each of at least some of the plurality of time points to obtain a plurality of dataset collections, the generating comprising: identifying, in the data associated with the subject, one or more datasets generated after the time point; and including the one or more datasets in the dataset collection; and processing the plurality of dataset collections using a second trained ML model to obtain a plurality of intervention responses, the processing comprising, for each of at least some of the plurality of dataset collections: generating a set of features using data from the dataset collection; and processing the set of features using the second trained ML model to obtain an intervention response of the plurality of intervention responses

Some embodiments provide a non-transitory computer-readable storage medium storing instructions. The instructions, when executed by at least one processor of a computer system, cause the at least one processor to perform a method for automatically extracting intervention responses from data associated with subjects. The method comprises: accessing imaging related data associated with a subject; processing the imaging related data using a first trained machine learning (ML) model to obtain a plurality of time points, the processing comprising: generating a plurality of sets of features using data from the image related data; and processing the plurality of sets of features using the first trained ML model to obtain the plurality of time points; identifying, from the data associated with the subject, a first collection of one or more datasets generated after a first one of the plurality of time points; generating, using the data associated with the subject, a dataset collection for each of at least some of the plurality of time points to obtain a plurality of dataset collections, the generating comprising: identifying, in the data associated with the subject, one or more datasets generated after the time point; and including the one or more datasets in the dataset collection; and processing the plurality of dataset collections using a second trained ML model to obtain a plurality of intervention responses, the processing comprising, for each of at least some of the plurality of dataset collections: generating a set of features using data from the dataset collection; and processing the set of features using the second trained ML model to obtain an intervention response of the plurality of intervention responses.

Some embodiments provide a computer system for automatically extracting intervention responses from data associated with subjects. The computer system comprises: at least one processor; and at least one non-transitory computer-readable storage medium storing instructions that, when executed by the at least one processor, cause the at least one processor to perform: accessing imaging related data associated with a subject; processing the imaging related data using a first trained machine learning (ML) model to obtain a plurality of time points, the processing comprising: generating a plurality of sets of features using data from the image related data; and processing the plurality of sets of features using the first trained ML model to obtain the plurality of time points; identifying, from the data associated with the subject, a first collection of one or more datasets generated after a first one of the plurality of time points; generating, using the data associated with the subject, a dataset collection for each of at least some of the plurality of time points to obtain a plurality of dataset collections, the generating comprising: identifying, in the data associated with the subject, one or more datasets generated after the time point; and including the one or more datasets in the dataset collection; and processing the plurality of dataset collections using a second trained ML model to obtain a plurality of intervention responses, the processing comprising, for each of at least some of the plurality of dataset collections: generating a set of features using data from the dataset collection; and processing the set of features using the second trained ML model to obtain an intervention response of the plurality of intervention responses.

Some embodiments provide a computer system for automatically extracting intervention responses from data associated with subjects. The computer system comprises a memory storing: parameters of at least one model trained to predict an intervention response; and at least one processor configured to: identify, from data associated with the subject, a first collection of one or more datasets; generate a first set of features using data from the first collection of one or more datasets; and process the first set of features using the parameters of the at least one model to obtain a first intervention response.

The foregoing is a non-limiting summary.

### BRIEF DESCRIPTION OF DRAWINGS

Various aspects and embodiments will be described with reference to the following figures. It should be appreciated that the figures are not necessarily drawn to scale. Items appearing in multiple figures are indicated by the same or a similar reference number in all the figures in which they appear.

FIG. 1A illustrates an example intervention response extraction system, according to some embodiments of the technology described herein.

FIG. 1B is a diagram illustrating an example interaction among the modules of the intervention response extraction system 100 of FIG. 1A, according to some embodiments of the technology described herein.

FIG. 1C is a diagram illustrating the extraction of another intervention response according to some embodiments of the technology described herein.

FIG. 1D is a diagram illustrating another example interaction among modules of the intervention response extraction system 100 of FIG. 1A, according to some embodiments of the technology described herein.

FIG. 2A is a diagram illustrating the determination of time points indicating predicted time periods in which an intervention response was determined for a subject, according to some embodiments of the technology described herein.

FIG. 2B is another diagram illustrating the determination of time points indicating predicted time periods in which an intervention response was determined for a subject, according to some embodiments of the technology described herein.

FIG. 3 is a diagram illustrating the extraction of intervention responses for a subject, according to some embodiments of the technology described herein.

FIG. 4A is a diagram of a machine learning model architecture 400 for extraction of intervention responses from data associated with a subject, according to some embodiments of the technology described herein.

FIG. 4B is a diagram of another machine learning model architecture 410 for extraction of intervention responses from data associated with a subject, according to some embodiments of the technology described herein.

FIG. 5A is an example process of extracting intervention responses for a subject from data associated with the subject, according to some embodiments of the technology described herein.

FIG. 5B is an example process of using data from a partition storing imaging related data to determine a time point, according to some embodiments of the technology described herein.

FIG. 6 is an example process of identifying dataset collections from which to extract intervention responses, according to some embodiments of the technology described herein.

FIG. 7 is an example process of determining an intervention response for a subject using data associated with the subject, according to some embodiments of the technology described herein.

FIG. 8 is an example process of filtering data extracted from a dataset collection (e.g., a collection of clinical documents), according to some embodiments of the technology described herein.

FIG. 9 is a diagram depicting filtering of data for a collection of datasets, according to some embodiments of the technology described herein.

FIG. 10 is a diagram of an illustrative computer system that may be used in implementing some embodiments of the technology described herein.

### DETAILED DESCRIPTION

The inventors have developed a computer intervention response extraction system. The intervention response extraction system automatically determines time points (e.g., dates) indicating when an intervention response was likely determined for a subject (e.g., by a physician) and uses those time points to identify data that is likely to indicate an intervention response of the subject. The intervention response extraction system then processes the identified data to automatically extract responses of the subject to intervention. As an illustrative example, the intervention response extraction system may process radiology report documents generated after radiographic imaging was performed on a subject to identify one or more dates indicating when an intervention response was likely determined for a subject. The intervention response extraction system may use the identified date(s) to identify clinical visit note document(s) stored in a time period following the identified date(s). The intervention response extraction system may process text from the clinical visit note document(s) to automatically extract intervention responses of the subject.

An important data point in real-world data (RWD) about a subject is the subject's response to an intervention (e.g., chemotherapy, vaccine, drug, surgery, or other intervention) for a condition (e.g., a solid tumor present in the subject, an infection, or other condition). This data point is a key measure of intervention efficacy and in regulatory approval of an intervention (e.g., as part of a clinical trial). However, conventional computer information extraction systems are unable to effectively extract intervention responses from clinical data. For example, in the case of solid tumors, the intervention response of a subject may be expressed using the Response Evaluation Criteria in Solid Tumors (RECIST) standard. According to this standard, a subject's response to intervention to treat a solid tumor may be a complete response (CR), a partial response (PR), stable disease (SD), and progressive disease (PD). Determining a subject's response using the RECIST standard involves analysis of radiographic images (e.g., magnetic resonance imaging (MRI) scans, X-Ray scans, computed tomography (CT) scans, and/or other radiographic images) that cannot be performed by a computer system. Moreover, even if a computer system could perform such analysis, radiographic images of a subject are rarely accessible. For example, radiographic images are typically not available in electronic health record (EHR) data of a subject that may be accessible by an information extraction system. Accordingly, conventional computer information extraction systems are unable to effectively extract intervention responses of a subject from available data associated with a subject.

Accordingly, the inventors have developed an intervention response extraction system that reliably extracts a subject's intervention responses from available data associated with the subject. The inventors recognized that certain datasets in data associated with a subject may contain information that indicates a subject's response to an intervention (e.g., as determined by a physician from analyzing radiographic image(s)). For example, one such dataset is a clinical visit note document that includes text indicating an intervention response determined by a clinician (e.g., a physician) based on the analysis of radiographic images. The intervention response extraction system may use a machine learning model with parameters trained to process text from the clinical visit note document(s) to output an intervention response indicated by the clinical visit note document(s). Continuing with the example of a tumor, the intervention response extraction system may process clinical visit note document(s) storing text indicating a physician's determination of a response to an intervention to treat the tumor.

Data associated with a subject may include hundreds or thousands of datasets (e.g., clinical visit note documents) and extraction of intervention responses may need to be performed for hundreds or thousands of subjects. Given this large amount of data, another challenge in extracting intervention responses of subjects is identifying which datasets are likely to contain information indicating intervention responses. Conventional information extraction systems are unable to identify specific datasets (e.g., specific clinical visit note document(s)) that are likely to indicate an intervention response of a subject. As a result, conventional information extraction systems need to expend a large amount of computing resources to process large amounts of data (e.g., clinical visit note documents) that do not include information about subjects' intervention responses. Moreover, conventional information extraction systems are unable to reliably extract the intervention responses of subjects from clinical data because of the inability to identify datasets that are likely to indicate the intervention responses of subjects.

Accordingly, the inventors have further developed an intervention response extraction system that can identify datasets, from among data associated with a subject, that are likely to include information indicating an intervention response of the subject. The inventors recognized that datasets (e.g., clinical visit note document(s)) indicating a subject's intervention response are often generated after radiographic imaging is performed on a subject. Accordingly, the inventors have developed an intervention response extraction system that determines time points (e.g., dates) indicating when an intervention response was determined for a subject. The intervention response extraction system uses imaging related data (e.g., radiology report documents) associated with the subject to identify time points indicating when an intervention response was likely determined for the subject.

The intervention response extraction system accesses imaging related data (e.g., radiology report documents) and processes the imaging related data (e.g., by processing text in radiology report document(s)) to predict time point(s) (e.g., assessment date(s)) when an intervention response was determined for a subject. Imaging related data may be generated after radiographic imaging is performed on a subject. For example, imaging related data may include imaging report documents (e.g., radiology report documents) generated after medical imaging (e.g., radiographic imaging) is performed on a subject. An imaging report document may indicate a time (e.g., a date) when an imaging test was performed, a type of imaging test performed (e.g., MRI scan, X-Ray scan, CT scan, or other type of scan), medical history of a subject (e.g., symptom(s), diagnosed condition(s)), findings in areas of the subject's body that were scanned, comparison of a finding with a finding from a previous imaging test, a summary of findings, diagnosis of a condition, recommendation for further testing, and/or other information. The intervention response extraction system uses the time point(s) to identify dataset(s) (e.g., clinical visit note document(s)) generated in the time period(s) following the time point(s). These dataset(s) are likely to include information indicating intervention responses of the subject. For example, dataset(s) immediately stored in a database (e.g., an EHR) following a time when radiographic imaging of a subject's tumor was performed has a high likelihood of including data (e.g., text) indicating an intervention response of the subject (e.g., determined by a physician). The intervention response extraction system uses the identified dataset(s) to extract the intervention responses of the subject.

In some embodiments, the intervention response extraction system uses a machine learning model to extract an imaging test date. The imaging test date may indicate a time period when an intervention response was determined for a subject and when dataset(s) (e.g., a clinical visit note documents) indicating the intervention response were generated (e.g., by adding new dataset(s) or updating existing dataset(s)). The machine learning model may be trained to process data (e.g., text) from imaging related data to output a predicted time point when a radiographic imaging test was performed on a subject. The intervention response extraction system may use the time point to identify dataset(s) (e.g., clinical visit note document(s)) from which to extract intervention response(s). The intervention response extraction system may use another machine learning model to extract an intervention response using the identified dataset(s). The machine learning model may be trained to process data (e.g., text) from the dataset(s) to output a predicted intervention response indicated in the dataset(s).

Some embodiments provide a computer system for automatically extracting intervention responses from data (e.g., clinical record data) associated with subjects. The computer system may be configured to: (1) access imaging related data (e.g., imaging report documents) associated with a subject; (2) process the imaging related data using a first trained machine learning (ML) model (e.g., a first neural network) to obtain a plurality of time points (e.g., dates indicating respective predicted time periods of intervention response determination for the subject). The computer system may be configured to process the imaging related data by: (1) generating a plurality of sets of features (e.g., numeric vectors) using data from the image related data; and (2) process the plurality of sets of features using the first trained ML model to obtain the plurality of time points. The computer system may be configured to generate, using the data associated with the subject, a dataset collection (e.g., collection of one or more clinical visit note documents) for each of at least some of the plurality of time points to obtain a plurality of dataset collections. The computer system may be configured to generate a dataset collection for each time point by: (1) identifying, in the data associated with the subject, one or more datasets (e.g., clinical visit note documents) generated after the time point; and (2) including the one or more datasets in the dataset collection. The computer system may be configured to: process the plurality of dataset collections using a second trained ML model to obtain a plurality of intervention responses (e.g., each selected from a group consisting of: complete response, partial response, stable disease, progressive disease, and unknown). The computer system may be configured to process each of at least some of the plurality of dataset collections by: (1) generating a set of features using data from the dataset collection; and (2) processing the set of features using the second trained ML model to obtain an intervention response of the plurality of intervention responses.

In some embodiments, the computer system is further configured to generate a dataset storing the at least some time points and corresponding intervention responses obtained using dataset collections generated using the at least some time points. In some embodiments, generating a dataset storing the at least some time points and corresponding intervention responses obtained using dataset collections generated using the at least some time points comprises generating a dataset storing: a first time point of the at least some time points; a first intervention response corresponding to the first time point, the first intervention response obtained using data from a first one of the plurality of dataset collections, the first dataset collection comprising one or more datasets generated after the first time point; a second time point of the at least some time points, the second time point subsequent to the first time point; and a second intervention response corresponding to the second time point, the second intervention response obtained using data from a second one of the plurality of dataset collections, the second dataset collection comprising one or more datasets generated after the second time point.

In some embodiments, the computer system may be configured to identify a subset of the plurality of time points such that each pair of the subset of time points is separated by at least a threshold amount of time (e.g., two weeks). In some embodiments, identifying the subset of time points comprises: identifying one or more of the plurality of time points that are less than the threshold amount of time after a respective preceding time point of the plurality of time points; and filtering out the one or more time points from the plurality of time points to obtain the subset of time points.

In some embodiments, the computer system may be further configured to: divide the imaging related data into a plurality of partitions; wherein generating the plurality of sets of features using the data from the image related data comprises using each of the plurality of partitions to generate a respective one of the plurality of sets of features. In some embodiments, the imaging related data comprises a plurality of imaging report documents; the plurality of partitions are associated with a respective plurality of time periods; and dividing the imaging related data into the plurality of partitions comprises: dividing the plurality of imaging report documents into the plurality of partitions by storing, in each of the plurality of partitions, one or more of the plurality of imaging report documents generated in a respective time period associated with the partition.

In some embodiments, identifying, in the data associated with the subject, one or more datasets generated after the time point comprises: identifying one or more carliest generated datasets (e.g., the three earliest generated datasets) after the time point.

In some embodiments, the computer system may be further configured to obtain the first trained ML model by performing training using training data comprising: sets of features generated using imaging related data associated with a plurality of subjects; and time point labels indicating target time point predictions for the sets of features. In some embodiments, the computer system may be further configured to obtain the second trained ML model by performing training using training data comprising: sets of features generated from datasets associated with a plurality of subjects; and intervention response labels indicating target intervention response predictions for the sets of features.

In some embodiments, generating the plurality of sets of features using data from the image related data comprises generating the plurality of sets of features using text extracted from the image related data. In some embodiments, generating the set of features using data from the dataset collection comprises: extracting text from the dataset collection; and generating the set of features using the text extracted from the dataset collection. In some embodiments, the text extracted from the dataset collection includes a first set of text and the computer system may be further configured to: extract a second set of text from one or more datasets generated before one or more datasets of the dataset collection; determine a measure of similarity between the first set of text and the second set of text; determine that the measure of similarity meets a threshold level of similarity; and remove the first set of text from the text extracted from the dataset collection when the measure of similarity meets the threshold similarity to obtain a filtered set of text; wherein generating the set of features using the text extracted from the dataset collection comprises generating the set of features using the filtered set of text.

The techniques described herein may be implemented in any of numerous ways, as the techniques are not limited to any particular manner of implementation. Examples of details of implementation are provided herein solely for illustrative purposes. Furthermore, the techniques disclosed herein may be used individually or in any suitable combination, as aspects of the technology described herein are not limited to the use of any particular technique or combination of techniques.

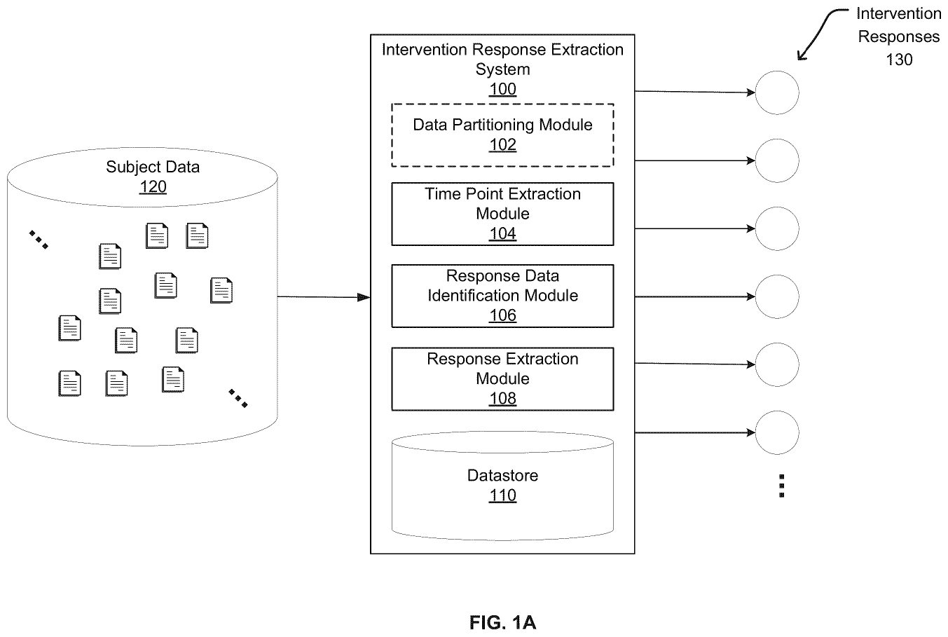

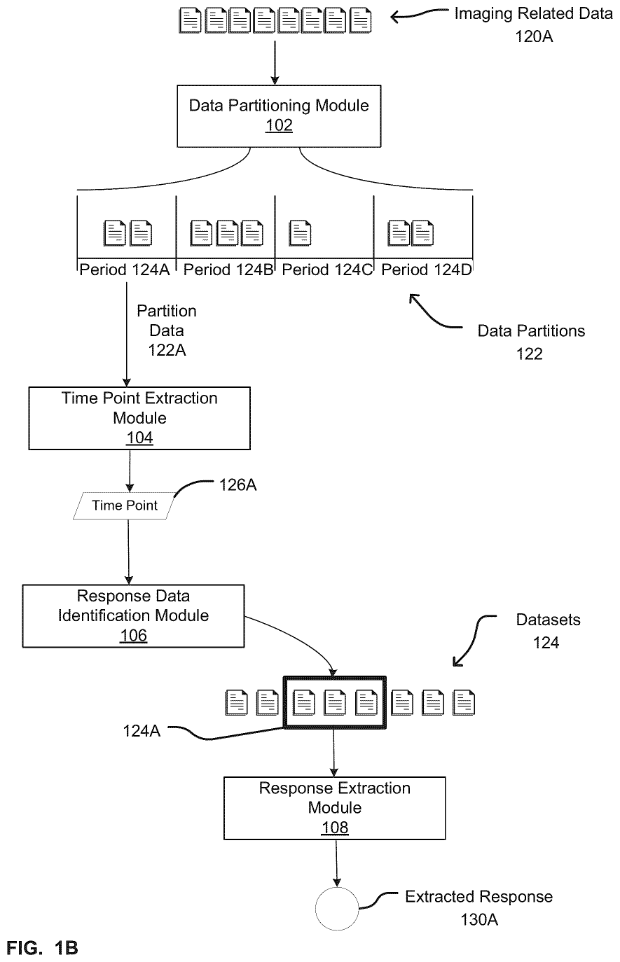

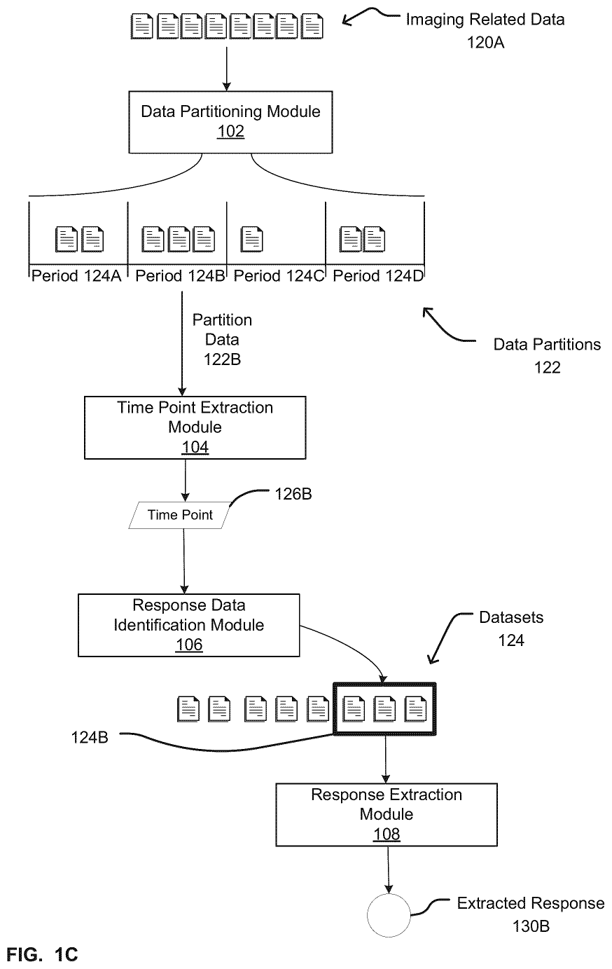

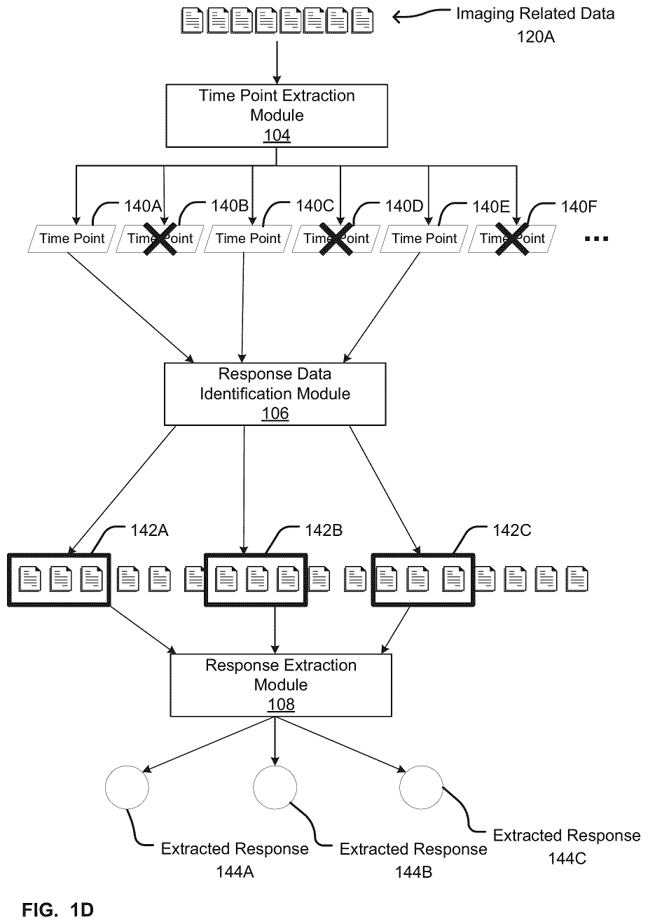

FIG. 1A illustrates an example intervention response extraction system 100, according to some embodiments of the technology described herein. As shown in the example of FIG. 1A, the intervention response extraction system 100 accesses data (e.g., clinical record data) associated with a given subject from a datastore 120. The intervention response extraction system 100 processes the data associated with the subject to automatically extract responses 130 of the subject to an intervention. For example, the intervention response extraction system 100 may process the data associated with the subject to automatically extract responses 130 to intervention performed for treatment of a tumor in the subject. As shown in FIG. 1A, the intervention response extraction system 100 includes multiple modules. The modules include a data partitioning module 102, a time point extraction module 104, a response data identification module 106, and a response extraction module 108. The intervention response extraction system 100 further includes a datastore 110.

In some embodiments, the intervention response extraction system 100 may access imaging related data associated with a subject from a datastore storing imaging related data associated with the subject that was generated at different times. In some embodiments, the intervention response extraction system 100 may access all imaging related data associated with a subject. In some embodiments, the intervention response extraction system 100 may access imaging related data associated with a subject that meets one or more criteria. For example, the intervention response extraction system 100 may filter imaging related data associated with a subject based on a category of a document, a title of a document, and/or a timestamp of the document. To illustrate, the intervention response extraction system 100 may access imaging related data with the following attributes: (1) a particular document category (e.g., “Radiology/Nuclear medicine”); (2) a title containing certain strings (e.g., “xr”, “ray”, “x-r”, “x-ray”, “ultra”, and/or “venous”); (3) a timestamp greater than or equal to a first date (e.g., a line of therapy start date) plus a number of days (e.g., 30 days) and less than or equal to a second date (e.g., a line of therapy end date).

In some embodiments, the intervention response extraction system 100 may access imaging related data that was generated during a particular time period. For example, the intervention response extraction system 100 may access imaging related data that was generated after the start date of a line of therapy (LOT) (e.g., chemotherapy, radiation, surgery, drug prescription, and/or a combination thereof) and before the end date of the LOT. The data partitioning module 102 may determine the start and end dates of a LOT from fields of clinical data records associated with the subject (e.g., by querying for values of the start and end dates of the LOT). As another example, the intervention response extraction system 100 may access imaging related data that was generated between the diagnosis date of a condition and the current date. In some embodiments, the intervention response extraction system 100 may access imaging related data that was generated in a user-specified time period. For example, the intervention response extraction system 100 may receive user input (e.g., through a graphical user interface (GUI)) indicating a time period (e.g., a date range) and access imaging related data generated (e.g., in clinical record data associated with the subject) in the time period. In some embodiments, the intervention response extraction system 100 may identify the imaging related data generated based on timestamps of datasets in the imaging related data. For example, intervention response extraction system 100 may access imaging related data with timestamps in a time period of 30-60 days, 60-90 days, 90-120 days, 120-150 days, 150-200 days, 200-250 days, 250-300 days, 300-360 days, or another suitable time period.

In some embodiments, the data partitioning module 102 may divide the accessed imaging related data into multiple partitions. A data partition may also be referred to as a “chunk”. The data partitioning module 102 may divide the imaging related data associated with the subject into partitions by organizing the data into the partitions based on times when the imaging related data was generated (e.g., as indicated by timestamps associated with the imaging related data). Each of the data partitions may include imaging related data (e.g., radiology report documents) generated in a respective time period associated with the data partition. In some embodiments, each partition may include imaging related data generated in a respective time period of at least 1-2 weeks, 2-4 weeks, 4-6 weeks, 6-8 weeks, 8-10 weeks, or another suitable amount of time. For example, each of the data partitions may include imaging related data generated in a time period of at least two weeks. In some embodiments, the data in a data partition may include data in addition to imaging related data, where the data is generated in a time period associated with the data partition. For example, the data in the data partition may include clinical visit note documents generated in the time period associated with the data partition (e.g., as indicated by timestamps of the clinical visit note documents).

In some embodiments, the imaging related data may include radiology report documents. The data partitioning module 102 may divide the radiology report documents into multiple partitions by: (1) sorting the radiology report documents based on time of generation (e.g., indicated by timestamps associated with the documents); and (2) dividing the radiology report documents into multiple data partitions associated with respective time periods. Each data partition may include radiology report documents generated in a time period associated with the data partition. In some embodiments, the time periods may be a sequence of consecutive time periods. In some embodiments, each of the time periods may be at least a threshold amount of time (e.g., 1 week, 2 weeks, 3 weeks, 4 weeks, 5 weeks, 6 weeks, or another suitable amount of time). As an illustrative example, the data partitioning module 102 may generate a first partition associated with a first time period by: (1) identifying a first radiology report document d₁; (2) including, in the first partition, the first radiology report document d₁ and all subsequent radiology report documents generated within the threshold amount of time from generation of the first radiology report document d₁. The data partitioning module 102 may generate a second partition associated with a second time period by: (1) identifying a second radiology report document d₂ that is the earliest radiology report document generated after the threshold amount of time from generation of the first radiology report document d₁; and (2) including, in the second data partition, the second radiology report document d₂ and all subsequent radiology report documents generated within the threshold amount of time from generation of the second radiology report document d₂. Thus, each data partition may include radiology report documents generated in an associated time period that has a length of at least the threshold amount of time.

In some embodiments, the data partitioning module 102 may allocate imaging related data to partition based on a time associated with the imaging related data. For example, the data partitioning module 102 may use timestamps included in datasets (e.g., radiology report documents) of the imaging related data to allocate the datasets into specific partitions. To illustrate, the data partitioning module 102 may access a timestamp of a stored radiology report document, identify a time period containing the time indicated by the timestamp, and allocate the radiology report document to a partition associated with the time period.

In some embodiments, the data partitioning module 102 may store imaging related data associated with the subject organized based on the partitions. For example, the data partitioning module 102 may store each partition as a row of a database table. To illustrate, each row of the table may store imaging related data with a timestamp in a time period defined by a start date and an end date. For example, a first row may store imaging related data with a timestamp indicating a date between Jan. 1, 2023, and Jan. 14, 2023. A second row may store imaging related data with a timestamp indicating a date between Jan. 15, 2023, and Jan. 30, 2023.

As indicated by the dashed lines around the data partitioning module 102 of FIG. 1A, in some embodiments, the intervention response extraction system 100 may not include a data partitioning module 102. In such embodiments, imaging related data associated with a subject may not be partitioned. The time point extraction module 104 may be configured to use the accessed imaging related data associated with a subject unpartitioned.

In some embodiments, the time point extraction module 104 may use imaging related data accessed by the intervention response extraction system 100 to determine time points (e.g., dates) indicating predicted time periods when intervention responses were determined for the subject. The time points may represent time periods after the time points in which an intervention response was determined for the subject. In some embodiments, the time point extraction module 104 may process imaging related data associated with a subject to obtain time points indicating predicted time periods of intervention response determination for the subject. The time point extraction module 104 may process the imaging related data by: (1) generating sets of features using data (e.g., text) from the imaging related data; and (2) processing the sets of features using a trained machine learning model to obtain the time points indicating the predicted time periods of intervention response determination. In some embodiments, each time point may indicate a predicted time period subsequent to the time point in which an intervention response was determined for the subject.

In some embodiments, the imaging related data associated with a subject may include multiple datasets and the time point extraction module 104 may determine a time point corresponding to each dataset. For example, each of the datasets may be an imaging report document (e.g., a radiology report document). The time point extraction module 104 may generate a set of features for each imaging report document (e.g., by extracting text from the imaging report document and generating the set of features using the extracted text). The time point extraction module 104 may process the set of features generated for each imaging report document using a trained machine learning model to obtain a respective time point. In some embodiments, the time point extraction module 104 may determine a time point for each dataset in imaging related data associated with a subject. In some embodiments, the time point extraction module 104 may determine a time point for each dataset in a subset of imaging related data associated with a subject. For example, the time point extraction module 104 may determine a time point for each dataset generated in a particular time period (e.g., between an LOT start and end date, a user-specified time period, or other time period).

In some embodiments, the time point extraction module 104 may use data partitions determined by the data partitioning module 102 to determine time points (e.g., dates) indicating predicted time periods when intervention responses were determined for the subject. The time point extraction module 104 may use data within a data partition to determine a time point corresponding to the data partition. The time point extraction module 104 may: (1) generate a set of features using data from the data partition; and (2) process the set of features using a trained machine learning model to obtain a time point. For example, the data partition may include unstructured data (e.g., text from radiology report document(s) and/or clinical visit note document(s)). The time point extraction module 104 may extract one or more snippets of text from the unstructured data and generate a set of features using the snippet(s) of text extracted from the unstructured data. As another example, the data partition may include structured data (e.g., values of fields in a database). The time point extraction module 104 may use structured data to generate the set of features. As another example, the time point extraction module 104 may use a combination of unstructured data (e.g., text snippets) and structured data (e.g., database field values) to generate the set of features.

In some embodiments, the machine learning model used by the time point extraction module 104 may be trained to output a time point. For example, the machine learning model may be trained to output a time point indicative of a predicted time period in which an intervention response was determined for the subject. In some embodiments, the machine learning model may output a classification indicating the time point. For example, the machine learning model may output scores associated with multiple different time points (e.g., dates). The time point extraction module 104 may output a time point with the greatest associated score as a time point of an intervention response determination. In some embodiments, the machine learning model may be a neural network model. The parameters of the neural network may be trained by applying a supervised learning technique (e.g., stochastic gradient descent) to a set of training data. For example, the training data may include sets of features and corresponding target time point labels. The neural network may be trained by applying stochastic gradient descent to the training data.

In some embodiments, the time point extraction module 104 may filter time points obtained using a trained machine learning model to obtain a subset of time points. In some embodiments, the time point extraction module 104 may obtain the subset of time points such that each pair of time points in the subset of time points is separated by at least a threshold amount of time. For example, each pair of time points in the subset of time points may be separated by at least 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 4 weeks, or other suitable amount of time. In one implementation, the time point extraction module 104 may identify a subset of time points in which each pair of time points is separated by at least 2 weeks. The time point extraction module 104 may identify the subset of time points by: (1) identifying time point(s) that are less than the threshold amount of time after a respective preceding time point in a set of time points obtained using the trained machine learning model; and (2) filtering out the identified time point(s) to obtain the subset of time points. The time point extraction module 104 may provide the subset of time points (e.g., a filtered set of time points) to the response data identification module 106 for identification of intervention response data.

In some embodiments, the response data identification module 106 may use time points determined by the time point extraction module 104 to identify intervention response data to use in determining an intervention response. In some embodiments, the response data identification module 106 may use a given time point to generate a corresponding dataset collection of one or more datasets to use in the extraction of an intervention response. The response data identification module 106 may identify, in data associated with a subject (e.g., clinical record data), dataset(s) generated after the time point, and include the identified dataset(s) in the dataset collection. For example, the response data identification module 106 may use the time point to identify one or more clinical visit note documents to use in extracting an intervention response. In some embodiments, the response data identification module 106 may identify a particular number of the earliest generated datasets generated after the time point and include the identified datasets in a dataset collection corresponding to the time point. In some embodiments, the response data identification module 106 may exclude datasets generated before or after the identified datasets from the dataset collection. For example, the response data identification module 106 may identify a particular number of clinical visit note documents (e.g., oncology clinical visit (OCV) note documents) generated after the time point (e.g., as indicated by timestamps of the clinical visit note documents). In some embodiments, the response data identification module 106 may identify the first 1, 2, 3, 4, 5, 6, 7, 8, 9, 10, or other suitable number of the earliest datasets generated after the time point. For example, the response data identification module 106 may identify the 3 earliest clinical visit note documents generated after a time point.

In some embodiments, the response data identification module 106 may store intervention response data associated with the subject organized based on time points. For example, the response data identification module 106 may store each dataset collection corresponding to a respective timepoint as a row of a database table. As an illustrative example, each row may store a dataset collection comprising the three earliest clinical visit note documents generated after a respective time point. The rows of the database table may be used to determine intervention responses for each time point.

In some embodiments, the response extraction module 108 may use intervention response data identified by the response extraction module 108 to determine the intervention responses 130. The response extraction module 108 may use a dataset collection identified using a time point to determine an extracted intervention response using data from the dataset collection. In some embodiments, the response extraction module 108 may generate a set of features (e.g., tokens) using data from the dataset collection and process the set of features using a trained machine learning model to output a corresponding extracted intervention response. For example, the response extraction module 108 may extract snippets of text from a set of clinical visit note document(s) in a dataset collection and generate a set of features using the snippets of text (e.g., by generating a set of tokens). The response extraction module 108 may process the set of features using the trained machine learning model to output an intervention response.

In some embodiments, the response extraction module 108 may use one or more previous intervention responses as input features. For example, in addition to tokens generated from text snippets extracted from clinical visit note document(s), the response extraction module 108 may include one or more values in a set of features that indicate previous intervention response(s) determined by the response extraction module 108 (e.g., for one or more predicted intervention response corresponding to time points that precede the time point for which an intervention response is being determined). The previous intervention response(s) may be metadata included as features for determining an intervention response. In some embodiments, the response extraction module 108 may filter out data (e.g., text snippets) that was included in dataset(s) generated prior to dataset(s) in a dataset collection corresponding to a time point. The data that was also included in previously generated dataset(s) may degrade prediction of an intervention response by the machine learning model (e.g., because the data may be unrelated to an intervention response determined in the time period after the time point).

In some embodiments, an intervention response determined by the response extraction module 108 may be one of multiple response categories. The machine learning model used by the response extraction module 108 may be trained to output a classification into one of the response types. For example, the machine learning model may output a score associated with each type and the response extraction module 108 may determine the response category with the highest score to be the extracted intervention response for a time point. To illustrate, the possible types for an intervention response for the treatment of a tumor may be a complete response (CR), partial response (PR), stable disease (SD), progressive disease (PD), or unknown. In some embodiments, the response extraction module 108 may be configured to use different sets of response categories for different types of conditions (e.g., different types of tumors, different pathogens, and/or other types of conditions).

In some embodiments, the response extraction module 108 may generate an output dataset including a set of time points (e.g., determined by the time point extraction module 104) and intervention responses corresponding to the time points (e.g., determined using dataset collections corresponding to the time points). For example, the output dataset may be a table storing time points and corresponding intervention responses. The table may include: (1) a first row storing a first time point and a corresponding first intervention response that was obtained using data from a first collection of dataset(s) generated after the first time point; and (2) a second row storing a second time point subsequent to the first time point and a corresponding second intervention response that was obtained using data from a second collection of dataset(s) generated after the second time point. In some embodiments, the response extraction module 108 may filter the output dataset to obtain a filtered output dataset. For example, the response extraction module 108 may filter the output dataset to include time points and corresponding intervention responses in a particular time period (e.g., between an LOT start and end date, a user-specified time period, or other time period).

In some embodiments, the datastore 110 of the intervention response extraction system 100 may store model parameters. The model parameters may include parameters of a machine learning model used by the time point extraction module 104 in predicting time points indicative of time periods in which an intervention response was determined for a subject. The model parameters may include parameters of a machine learning model used by the response extraction module 108 in predicting intervention responses. The datastore 110 stores extraction data. The extraction data may include data partitions, time points, dataset collections identified by the response data identification module 106, sets of features (e.g., generated by the time point extraction module 104 and/or the response extraction module 108), and/or other data used in extracting the intervention responses 130. In some embodiments, the datastore 110 may store output dataset(s) generated by the response extraction module 108 (e.g., storing time points and corresponding intervention responses for a subject).

In some embodiments, the datastore 110 may comprise data storage hardware storing the model parameters and extraction data. For example, the datastore 110 may comprise one or more hard drives storing the data associated with the subjects. In some embodiments, the datastore 110 may be a distributed database storing the data.

In some embodiments, the intervention response extraction system 100 may access data associated with a subject from the datastore 120. The datastore 120 may store data associated with multiple different subjects including the subject for whom intervention response extraction is being performed. For example, the datastore 120 may store clinical record data associated with different subjects (e.g., accessed from an electronic health record (EHR) database). Data associated with a subject that is stored in the datastore 120 may include imaging related data, textual notes, medical history data, immunizations, laboratory data, medical claims data, and/or other data related to the subject's health. For example, the data associated with the subject may include radiology report documents generated after the performance of radiographic imaging on the subject (e.g., MRI, X-Ray, CT, ultrasound, and/or other radiographic imaging). As another example, the data associated with the subject may include clinical visit note documents. A clinical note may, for example, include text describing a diagnosis or intervention response determined by a clinician (e.g., a physician). To illustrate, a clinical note may include text describing a response of a subject to intervention for the treatment of a tumor as determined by a clinician.

In some embodiments, the datastore 120 may comprise data storage hardware storing the data associated with the subjects. For example, the datastore 120 may comprise one or more hard drives storing the data associated with the subjects. In some embodiments, the datastore 120 may be a database storing the data associated with the subjects. The database may be a queryable database storing the data associated with the subjects. Although in the example embodiment of FIG. 1A the datastore 120 is shown separate from the intervention response extraction system 100, in some embodiments, the datastore 120 may be part of the intervention response extraction system 100.

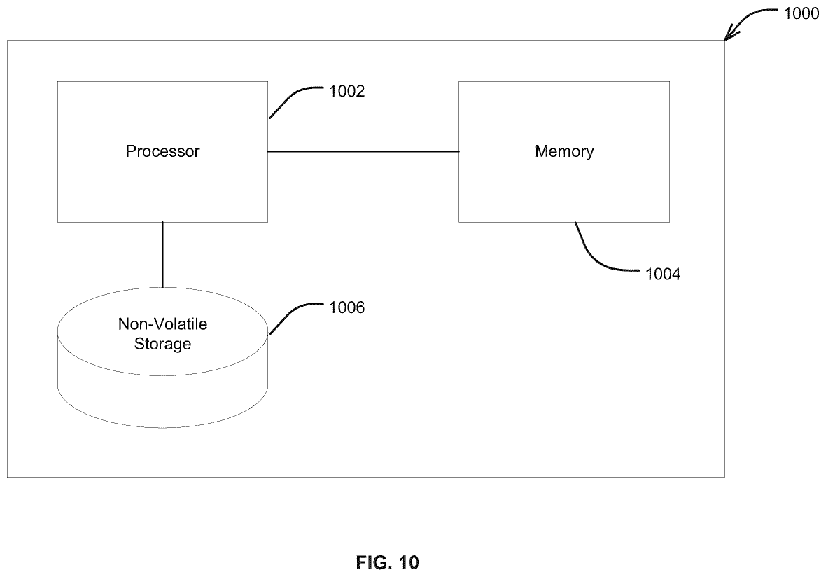

The intervention extraction system 100 may be implemented using a suitable computer system. For example, the intervention extraction system 100 may be implemented using the computer system 1000 described herein with reference to FIG. 10. In some embodiments, the modules 102, 104, 106, 108 may be implemented as respective sets of executable instructions stored in memory of the computer system. In some embodiments, the datastore 110 may be memory of the computer system.

FIG. 1B is a diagram illustrating an example interaction among the modules 102, 104, 106, 108 of the intervention response extraction system 100 of FIG. 1A, according to some embodiments of the technology described herein. The example of FIG. 1B shows how an intervention response 130A of the intervention responses 130 may be extracted for a subject.

In the example of FIG. 1B, the intervention response extraction system accesses a set of imaging related data 120A (e.g., radiology report documents) associated with a subject (e.g., from datastore 120). In some embodiments, the imaging related data 120A may include all the imaging related data associated with a subject. In some embodiments, the imaging related data 120A may be a subset of imaging related data associated with the subject. The intervention response extraction system 100 may access a subset of imaging related data stored during a particular time period. In some embodiments, the particular time period may be specified by user input (e.g., through a GUI). In some embodiments, the particular time period may be automatically determined by the intervention response extraction system 100. For example, the intervention response extraction system 100 may: (1) determine a time point (e.g., a date) when the subject was diagnosed with a condition (e.g., a tumor); and (2) select a subset of imaging related data associated with the subject generated after the time point.

The data partitioning module 102 partitions the imaging related data 120A. As illustrated in FIG. 1B, the data partitioning module 102 determines multiple data partitions 122 each associated with a respective one of time periods 124A, 124B, 124C, 124D. In some embodiments, the periods 124A, 124B, 124C, 124D may be time periods of at least a minimum amount of time (e.g., of at least two weeks). Each partition may include imaging related data generated during its associated time period (e.g., as indicated by timestamps associated with the data). In some embodiments, the data partitioning module 102 may include, in each data partition, data in addition to the imaging related data generated in its associated time period. For example, the additional data may include clinical visit note documents generated in the time period and/or other clinical data generated during the time period.

The time point extraction module 104 accesses data from a first partition associated with time period 124A. For example, the time point extraction module 104 may access imaging related data in the first partition. As another example, the time point extraction module 104 may access clinical visit note documents in the first partition in addition to the imaging related data. The time point extraction module 104 uses the partition data 122A to determine a time point 126A of an intervention response determination. The time point extraction module 104 may use the partition data 122A to: (1) generate a set of features (e.g., by extracting text snippets from the partition data 122A and generating a set of tokens therefrom as the set of features); and (2) provide the set of features as input to a trained machine learning model to obtain output indicating the time point 126A. In some embodiments, the time point 126A may indicate a predicted time period in which an intervention response was determined for the subject.

As shown in FIG. 1B, the response data identification module 106 accesses the time point 126A. The response data identification module 106 may use the time point 126A to identify a dataset collection 124A from among datasets 124 associated with the subject to use in extracting an intervention response (e.g., to include in a dataset collection). For example, the identified dataset collection 124A may be those datasets that the response data identification module 106 has determined have a high likelihood of including data indicating an intervention response of the subject (e.g., determined by a clinician). In some embodiments, the datasets 124 may include clinical visit note documents associated with the subject. The response data identification module 106 may use the time point 126A to identify a subset of the clinical visit note documents to use in extracting an intervention response. In some embodiments, the response data identification module 106 may identify a certain number of earliest clinical visit note documents generated after the time point 126A and include the identified clinical visit note documents in the dataset collection 124A. For example, the response data identification module 106 may identify the three earliest clinical visit note documents generated after the time point 126A (e.g., as indicated by time points associated with the clinical visit note documents).

The response extraction module 108 may use the dataset collection 124A (e.g., collection of clinical visit note documents) generated by the response data identification module 106 to determine an extracted response 130A of the subject. The response extraction module 108 may use datasets in the dataset collection 124A to: (1) generate a set of features (e.g., by extracting text snippets and generating a set of tokens therefrom as the set of features); and (2) providing the set of features as input to a trained machine learning model to obtain output indicating the extracted response 130A (e.g., a classification into one of multiple response categories). The response extraction module 108 may store the extracted response 130A (e.g., in an output dataset and/or the datastore 110).

FIG. 1C is a diagram illustrating extraction of another intervention response 130B, according to some embodiments of the technology described herein. As shown in FIG. 1C, the time point extraction module 104 access data 122B from a second partition associated with time period 124B and uses the data 122B to determine a time point 126B indicating a predicted time period in which an intervention response was determined for the subject. The response data identification module 106 accesses the time point 126B and uses it to generate dataset collection 124B from among the datasets 124 associated with the subject (e.g., by identifying the three earliest clinical visit note documents generated after the time point 126B and including them in the dataset collection 124B). The response extraction module 108 uses the datasets in the dataset collection 124B to determine the extracted response 130B.

It should be appreciated that although the response extractions illustrated in FIGS. 1B and 1C are illustrated as occurring sequentially, in some embodiments, the responses 130A, 130B may be extracted in parallel. Partition data 122A and partition data 122B may be processed in parallel to obtain time points 126A, 126B. The time points 126A, 126B may be used by the response data identification module 106 in parallel to identify dataset collections 124A, 124B. The response extraction module 108 may determine the extracted responses 130A, 130B in parallel using the identified dataset collections 124A, 124B.

FIG. 1D is a diagram illustrating another example interaction among modules of the intervention response extraction system 100 of FIG. 1A, according to some embodiments of the technology described herein. In the example of FIG. 1D, the time point extraction module 104 uses imaging related data 120A accessed by the intervention response extraction system 100 to determine time points indicating time periods of intervention response determination for a subject. The time points include time points 140A, 140B, 140C, 140D, 140E, 140F. In the example embodiment of FIG. 1D, the imaging related data 120A is not partitioned. As such, the example interaction of FIG. 1D may occur without use of the data partition module 102. For example, the time point extraction module 104 may determine a time point for each of the datasets (e.g., radiology report documents) included in the imaging related data 120A (e.g., by extracting text from the dataset and processing the text using a trained machine learning model to obtain the corresponding time point).

As illustrated in the example of FIG. 1D, the time point extraction module 104 further filters the time points to generate a subset of time points that are used by the response data identification module 106. Example techniques of filtering the time points are described herein with reference to FIG. 1A. For example, the time point extraction module 104 may filter the time points 140A, 140B, 140C, 140D, 140E, 140F such that each pair of time points in the subset of time points is separated by at least two weeks. In the example of FIG. 1D, the time point extraction module 104 filters out time points 140B, 140D, 140F (e.g., because they are within two weeks of respective preceding time points 140A, 140C, 140E). The filtered set of time points 140A, 140C, 140E may be provided to the response data identification module 106.

As illustrated in the example of FIG. 1D, the response data identification module 106 uses the subset of time points 140A, 140C, 140E to identify respective dataset collections 142A, 142C, 142E. For example, the response identification module 106 may identify the three earliest generated datasets after each of the time points 142A, 142C, 142E and include the identified datasets in respective dataset collections 142A, 142B, 142C. The response extraction module 108 uses the identified dataset collections 142A, 142B, 142C to determine respected extracted intervention responses 144A, 144B, 144C (e.g., by generating a set of features using data from each dataset collection and processing the set of features using a trained machine learning model to obtain a corresponding intervention response). The response extraction module 108 may use dataset collection 142A to obtain extracted response 144A, dataset collection 142B to obtain extracted response 144B, and dataset collection 142C to obtain extracted response 144C.

It should be appreciated that although the response extraction illustrated in FIG. 1D are illustrated as occurring in parallel, in some embodiments, the responses 144A, 144B, 144C may be extracted sequentially. Imaging related data may be processed sequentially to obtain time points 140A, 140B, 140C, 140D, 140E, 140F. The time points 140A, 140C, 140E may be used by the response data identification module 106 sequentially to identify dataset collections 142A, 142B, 142C. The response extraction module 108 may determine the extracted responses 144A, 144B, 144C sequentially using the identified dataset collections 144A, 144B, 144C.

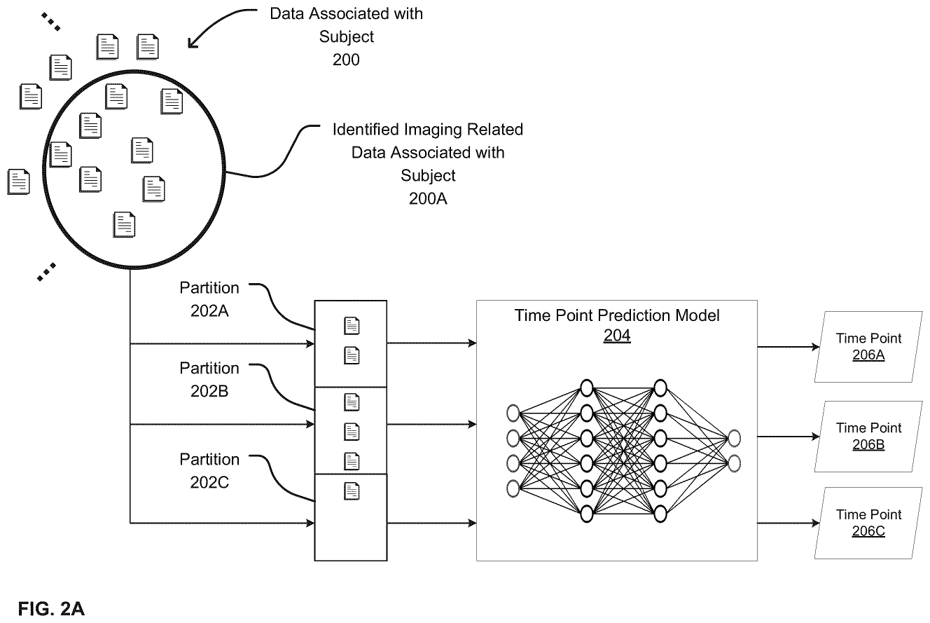

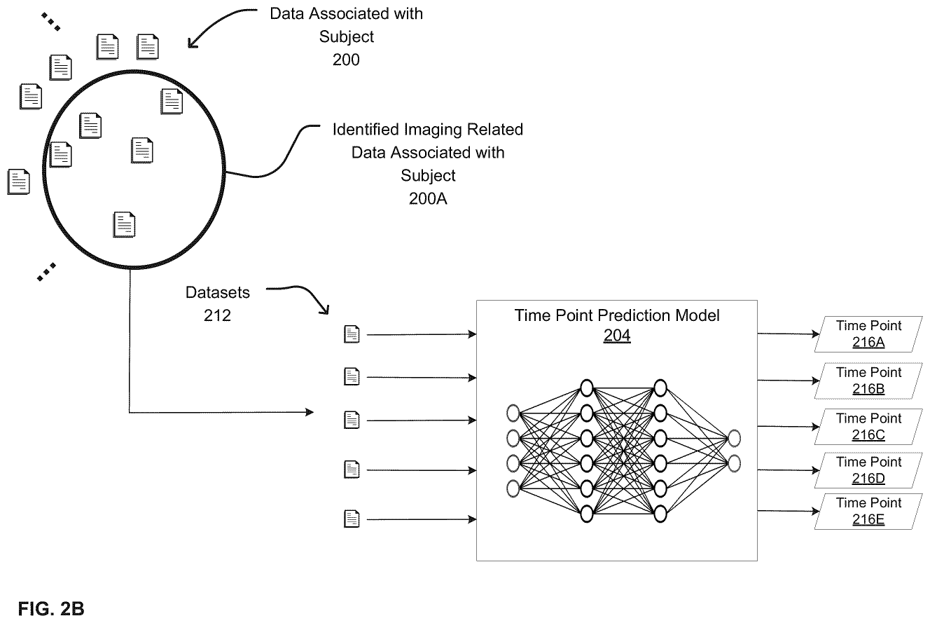

FIG. 2A is a diagram illustrating the determination of time points 206A, 206B, 206C in which an intervention response was determined for a subject, according to some embodiments of the technology described herein. The determination illustrated in FIG. 2A may be performed by the intervention extraction system 100 of FIG. 1A. For example, the determination may be performed by the intervention extraction system 100 using the data partitioning module 102 and time point extraction module 104.

As shown in FIG. 2A, the system access data 200 associated with a subject (e.g., clinical record data from an EHR). The data 200 may include imaging related data (e.g., radiology report documents) associated with the subject. The system may identify a portion 200A of the imaging related data to use in determining the time points 206A, 206B, 206C. In some embodiments, the system may identify imaging related data generated in a particular time window (e.g., a certain number of days in the past, a number of days after diagnosis of a condition, or a specific user-specified date range). For example, the system may identify radiology report documents with a timestamp within the particular time window as the imaging related data 200A.

After identifying the imaging related data 200A, the system partitions the data 200A into multiple partitions 202A, 202B, 202C. The system may generate the partitions 202A, 202B, 202C such that each partition represents a respective time period. In some embodiments, the time periods may be of equal length (e.g., two weeks). Each of the partitions 202A, 202B, 202C may include imaging related data generated in the time period that the partition represents. For example, the partition 202A may include radiology report documents with a timestamp in a first two-week period, the partition 202B may include radiology report documents with a timestamp in a second two-week period subsequent to the first two-week period, and the partition 202C may include radiology report documents with a timestamp in a third two-week period subsequent to the second two-week-period. In some embodiments, the system may further include, in each of the partitions 202A, 202B, 202C, data in addition to the imaging related data of the partition. For example, the system may include documents (e.g., clinical visit note documents) generated in a time period represented by the partition.

The system may use the data of each of the partitions 202A, 202B, 202C to determine a corresponding one of the time points 206A, 206B, 206C. The system may use a time point prediction model 204 to predict time points 206A, 206B, 206C. As indicated by the arrows between the partitions 202A, 202B, 202C and the time point prediction model 204, the system may use data from each of the partitions to generate a set of features to provide as input to the time point prediction model 204. For example, the system may extract text snippets from one or more radiology report documents by identifying keyword(s) in the radiology report documents and extracting segments of text (e.g., a predefined number of words or characters) following and/or preceding the keyword(s). Example keywords that may be identified in imaging related data (e.g., radiology report documents) include “imaging”, “images”, “scan”, “pet”, “ct”, “mri”, “evidence”, “response”, “cr”, “remission”, “stable”, “larger”, “bigger”, “new”, “worsen”, “progress”, and/or other keywords. The system may tokenize the extracted text snippets to obtain a token vector. The system may further determine a numeric representation of the token vector. For example, the system may determine a word embedding for each token that is a vector of real number values (e.g., a vector of 128 real numbers) representing the token. The numeric representation of the token vector (e.g., a numeric vector) may form a set of features that the system may input to the time point prediction model 204.

In some embodiments, the time point prediction model 204 may be a machine learning model trained to predict a time point (e.g., a date) for an input set of features. The predicted time point may indicate a predicted time period in which an intervention response was determined for the subject. For example, the input set of features may be a real value embedding of a vector (e.g., a token vector) generated using text snippets extracted from a data partition. The machine learning model may be trained using training data. The training data may include sets of input features and labels indicating target time points for the sets of input features. For example, the training data may include: (1) sets of features generated from sets of radiology report documents generated in a time period (e.g., a period of at least two weeks); and (2) corresponding date labels that are to be output by the machine learning model for each set of features. A date label corresponding to a set of radiology report documents may be a predetermined date of an intervention response determination (e.g., performed by a physician) after generation of the set of radiology report documents. For example, the date labels may be dates manually extracted by analyzing sets of radiology report documents. In some embodiments, each set of features in the training data may also be generated using clinical visit note documents generated in a time period in which a set of radiology report documents were generated. For example, a set of features may be generated using text from a set of radiology report documents generated in a two-week period and clinical visit note documents generated in the two-week period.

In some embodiments, the machine learning model may be trained by applying a supervised learning technique (e.g., stochastic gradient descent) to a set of training data. The machine learning model may be trained by: (1) providing the sets of features as inputs to the machine learning model to obtain time point predictions; (2) comparing the time point to the target time points; and (3) updating parameters of the machine learning model based on a difference between the time points and the target time points. In some embodiments, the supervised learning technique may be an iterative technique performed to minimize a loss function. For example, stochastic gradient descent may be applied to minimize a loss function based on a difference between the time points and the target time points.

In some embodiments, the time point prediction model 204 may include a neural network, a logistic regression model, a linear regression model, a regression model, a random forest model, a K-Nearest Neighbor (KNN) model, a K-Means model, a decision tree model, a cox proportional hazards regression model, a Naïve Bayes model, a support vector machines (SVM) model, and/or other suitable machine learning model. For example, the time point prediction model 204 may be a neural network model trained to predict a time point for an input set of features. The neural network may include one or more recurrent neural network (RNN) layers, one or more long short-term memory network (LSTM) layers, one or more convolutional neural network (CNN) layers, one or more feedforward neural network layers, and/or one or more other suitable neural network layers of another type. The system may provide the set of features (e.g., a vector or matrix of values) as input to the neural network to obtain a corresponding output indicating a time point. In some embodiments, the output may indicate a classification result. For example, the output may include probabilities associated with various different possible time points (e.g., dates in a year). The system may determine the time point to be the one associated with the greatest probability. For example, the time point prediction model 204 may be trained to receive, as input, a tokenized snippet vector and output probabilities that an intervention response was determined at different time points (e.g., dates). Example machine learning models that may be used for the time point prediction model 204 are described in U.S. Patent Application Publication No. 2022/0284999 published on Sep. 8, 2022, which is incorporated by reference herein in its entirety.

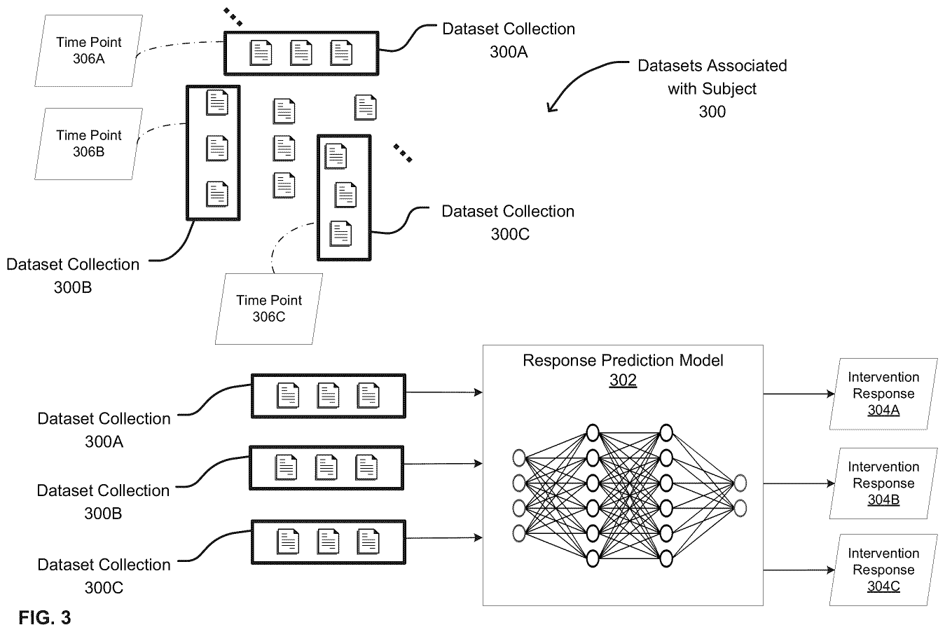

As shown in the example of FIG. 2A, the system determines a time point for each of the data partitions 202A, 202B, 202C. The system determines time point 206A using data from partition 202A, time point 206B using data from partition 202B, and time point 206C using data from partition 202C. The time points 206A, 206B, 206C may subsequently be used in extracting intervention responses of the subject (e.g., as described herein with reference to FIG. 3).

As an illustrative example, the identified imaging related data 200A associated with the subject may be radiology report documents generated after radiographic imaging performed on a subject for a tumor. Each of the partitions 202A, 202B, 202C may include radiology report documents generated during the time period that the partition represents. In some embodiments, the partitions 202A, 202B, 202C may each include clinical visit note documents generated during the time period represented by the time period. For each of the partitions 202A, 202B, 202C, the system may generate a corresponding set of features and provide the set of features as input to the time point prediction model 204 to obtain time points 206A, 206B, 206C. The time points 206A, 206B, 206C may indicate predicted time periods in which an intervention response was determined for the subject (e.g., by a physician).

FIG. 2B is another diagram illustrating the determination of time points 216A, 216B, 216C, 216D, 216E indicating predicted time periods in which an intervention response was determined for a subject, according to some embodiments of the technology described herein. The determination illustrated in FIG. 2B may be performed by the intervention extraction system 100 of FIG. 1A. For example, the determination may be performed by the intervention extraction system 100 using the time point extraction module 104. In contrast to the example embodiment of FIG. 2A, in the example embodiment of FIG. 2B, imaging related data 200A is not partitioned prior to determination of time points using the imaging related data 200A.

The imaging related data 200A may be identified from the data associated with the subject 200 as described herein with reference to FIG. 2A. As shown in FIG. 2B, the data 200A includes datasets 212 (e.g., imaging report documents) that are each processed using the time point prediction model 204. The system may use data (e.g., extracted text) from each of the datasets 212 to generate a set of features to provide as input to the time point prediction model 204. For example, the system may extract text snippets from one of the datasets 212 by identifying keyword(s) in the dataset and extracting segments of text (e.g., a predefined number of words or characters) following and/or preceding the keyword(s). Example keywords that may be identified in a dataset include “imaging”, “images”, “scan”, “pet”, “ct”, “mri”, “evidence”, “response”, “cr”, “remission”, “stable”, “larger”, “bigger”, “new”, “worsen”, “progress”, and/or other keywords. The system may tokenize the extracted text snippets to obtain a token vector. The system may further determine a numeric representation of the token vector. For example, the system may determine a word embedding for each token that is a vector of real number values (e.g., a vector of 128 real numbers) representing the token. The numeric representation of the token vector (e.g., a numeric vector) may form a set of features that the system may input to the time point prediction model 204.

As shown in the example embodiment of FIG. 2B, the system obtains time points 216A, 216B, 216C, 216D, 216E using the time point prediction model 204. In some embodiments, each of the time points 216A, 216B, 216C, 216D, 216E may be obtained using a respective set of features generated using data from a respective one of the datasets 212. Although not illustrated in FIG. 2B, in some embodiments, the system may filter the time points 216A, 216B, 216C, 216D, 216E to obtain a subset of time points (e.g., as illustrated in the example of FIG. 1D). The subset of time points may be used to identify data to use in extracting intervention responses for a subject.

FIG. 3 is a diagram illustrating the extraction of intervention responses 304A, 304B, 304C for a subject, according to some embodiments of the technology described herein. The extraction illustrated in FIG. 3 may be performed by the intervention extraction system 100 of FIGS. 1A-1B. For example, the prediction may be performed by the intervention extraction system 100 using response data identification module 106 and response extraction module 108.

In the example of FIG. 3, the system uses the time points 306A, 306B, 306C to identify datasets from which to extract intervention responses of a subject. As shown in FIG. 3, the system has access to datasets 300 associated with the subject. For example, the datasets 300 may be clinical visit note documents associated with the subject. As another example, the datasets 300 may be clinical visit note documents with a particular attribute (e.g., clinical visit note documents with a particular category tag). The system uses the time points 306A, 306B, 306C to identify respective subsets of the datasets 300 associated with the subject. In some embodiments, the system may identify a particular number of the earliest generated datasets after the time point. For example, the system may identify the three earliest generated datasets after the time point. In some embodiments, the system may identify all the datasets generated within a time window after the time point. For example, the system may identify all the datasets generated within a 1-week, 2-week, 3-week, or 4-week window after the time point. The system generates a dataset collection corresponding to each of the time points 306A, 306B, 306C. Each dataset collection may include the datasets identified based on a respective time point (e.g., the three earliest datasets generated after the time point). In the example of FIG. 3, the system has generated dataset collection 300A based on time point 306A, dataset collection 300B based on time point 306B, and dataset collection 300C based on time point 206C. For example, each of the dataset collections 300A, 300B, 300C may be a set of clinical visit note documents identified based on respective time points 306A, 306B, 306C.

The system may use the data of each of dataset collections 300A, 300B, 300C to extract a corresponding one of the intervention responses 304A, 304B, 304C. The system may use a response prediction model 302 to determine the intervention responses 304A, 304B, 304C. As indicated by the arrows between the dataset collections 300A, 300B, 300C and the response prediction model 302, the system may use data from each of the datasets to generate a set of features to provide as input to the response prediction model. For example, the system may extract text snippets from one or more clinical visit note documents by identifying keyword(s) in the clinical visit note document(s) and extracting segments of text (e.g., a predefined number of words or characters) following and/or preceding the keyword(s). Example keywords that may be identified include “response”, “remission”, “stable”, “larger”, “bigger”, “new”, “worsen”, “progress”, and/or other keywords. The system may tokenize the extracted text snippets to obtain a token vector. The system may further determine a numeric representation of the token vector. For example, the system may determine a word embedding for each token that is a vector of real number values (e.g., a vector of 128 real numbers) representing the token. The numeric representation of the token vector (e.g., a numeric vector) may form a set of features that the system may input to the response prediction model 302.

The system may apply filtering to text snippets extracted from a dataset collection prior to generating a set of features. In some embodiments, the system may filter out text snippets that appeared in a previously processed dataset collection. For example, clinical visit note documents may include text copied from previous clinical visit note documents (also referred to as “copy forward text”). Accordingly, a text snippet extracted from a collection of clinical visit note documents may have been included in another set of clinical visit note documents that had been previously used to generate a set of features. The system may filter out such text snippets such that they are not used to generate a set of features as these text snippets are unlikely to include information about a current intervention response determination. As such, such text snippets may degrade the accuracy of intervention response predictions made by the response prediction model 302. In some embodiments, the system may determine a measure of similarity between text snippets extracted from a dataset collection and text snippets extracted from previously processed dataset collection(s). For example, the system may determine the measure of similarity to be a percentage of overlap between text snippets. The system may remove a text snippet with a threshold measure of similarity (e.g., a threshold percentage of overlap) with a text snippet extracted from a previously processed dataset collection. The system may remove a text snippet with a measure of similarity relative to a text snippet extracted from a previously processed dataset collection of at least 50-60%, 60-70%, 70-80%, 80-90%, 90-100%, or other suitable similarity threshold. For example, the system may remove text snippets with a measure of similarity of at least 80% relative to a text snippet extracted from a previously processed dataset collection.

In some embodiments, the response prediction model 302 may be a machine learning model trained to output a predicted intervention response for an input set of features. For example, the input set of features may be a real value embedding of a vector (e.g., a token vector) generated using text snippets extracted from a dataset collection. The machine learning model may be trained using training data. The training data may include sets of input features and labels indicating target intervention responses for the sets of input features. For example, the training data may include: (1) sets of features generated from a dataset collection (e.g., a collection of clinical visit note documents); and (2) corresponding intervention response labels for each set of features. An intervention response label corresponding to a collection of clinical visit note documents may be a predetermined intervention response. For example, the intervention response may be manually extracted by analyzing the clinical visit note documents. In some embodiments, each set of features in the training data may include an indication of a previously determined intervention response. For example, a set of features may be generated using clinical visit note documents generated after a certain date, and may include an indication of the earliest intervention response determined prior to the date.

In some embodiments, the machine learning model may be trained by applying a supervised learning technique (e.g., stochastic gradient descent) to a set of training data. The machine learning model may be trained by: (1) providing the sets of features as inputs to the machine learning model to obtain predicted intervention responses; (2) comparing the predicted intervention responses to the label intervention responses; and (3) updating parameters of the machine learning model based on a difference between the predicted intervention responses and the target intervention responses. In some embodiments, the supervised learning technique may be an iterative technique performed to minimize a loss function. For example, stochastic gradient descent may be applied to minimize a loss function based on a difference between the predicted intervention responses and the label intervention responses.

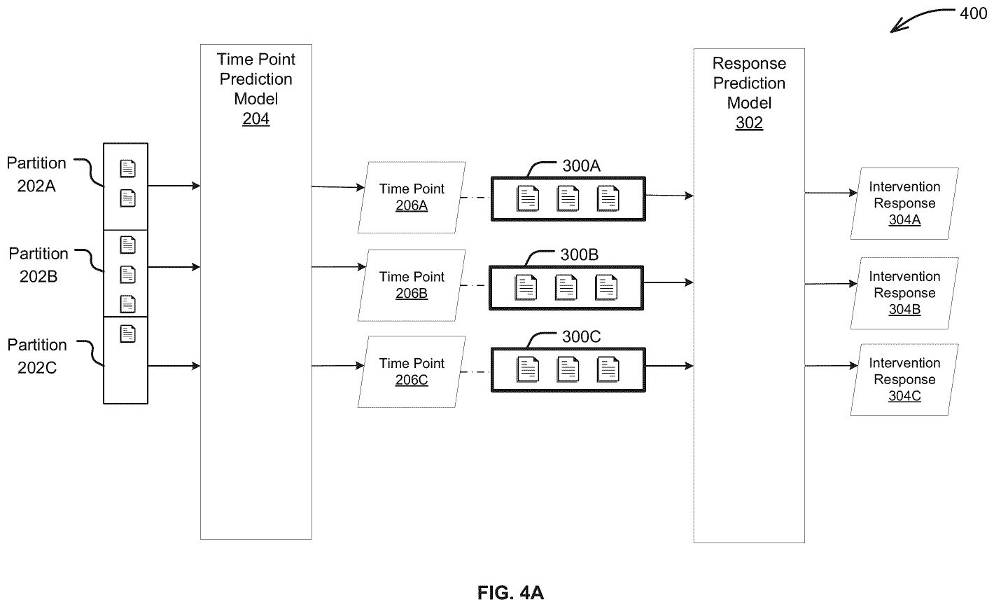

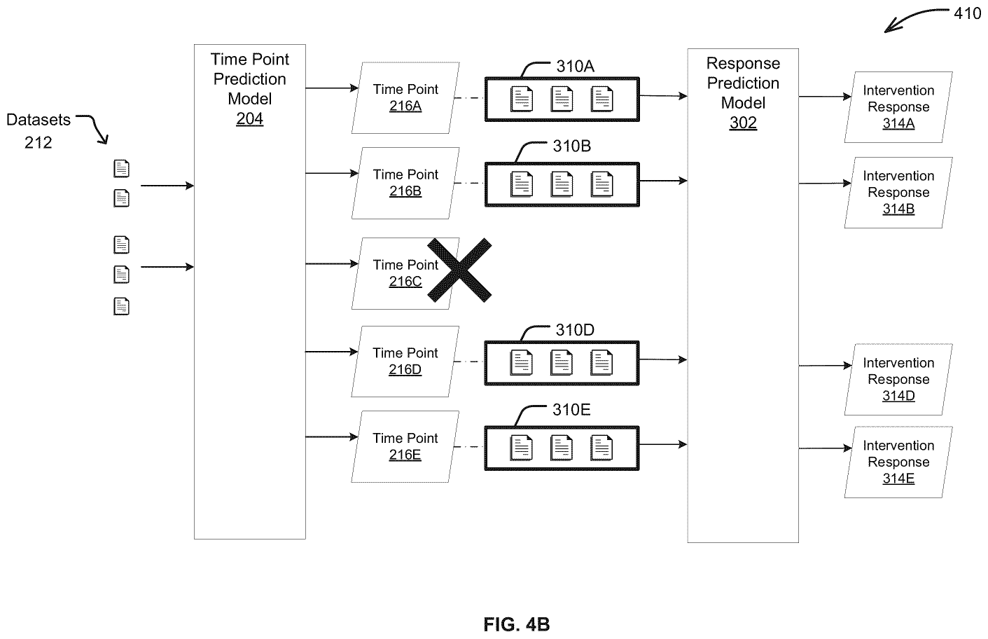

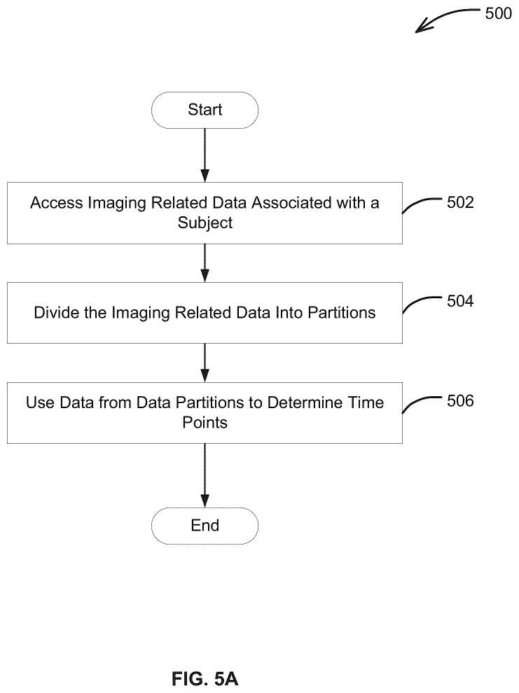

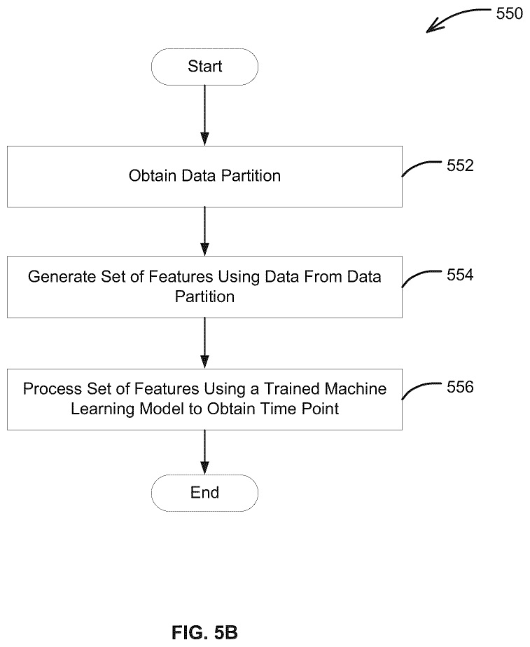

In some embodiments, the response prediction model 302 may include a neural network, a logistic regression model, a linear regression model, a regression model, a random forest model, a K-Nearest Neighbor (KNN) model, a K-Means model, a decision tree model, a cox proportional hazards regression model, a Naïve Bayes model, a support vector machines (SVM) model, and/or other suitable machine learning model. For example, the response prediction model 302 may be a neural network model trained to predict a time point for an input set of features. For example, the neural network may include a recurrent neural network (RNN), a long short-term memory network (LSTM), a convolutional neural network (CNN), a feedforward neural network, and/or another type of neural network. The system may provide the set of features (e.g., a vector or matrix of values) as input to the neural network to obtain a corresponding output indicating a predicted intervention response. In some embodiments, the output may indicate a classification result. For example, the output may include probabilities associated with various different intervention response categories. The system may determine the predicted intervention response to be the response category associated with the greatest probability. Example machine learning models that may be used for the response prediction model 302 are described in U.S. Patent Application Publication No. US 2021/0027894 published on Jan. 28, 2021, which is incorporated by reference herein. Descriptions of example machine learning models are provided in the patent application publication with reference to FIGS. 4A, 4B and 5 of the patent application publication and are incorporated herein by reference. Example techniques of using a machine learning model are described in paragraphs 57-59 of the patent application publication.

As shown in the example of FIG. 3, the system determines an intervention response for each of the dataset collections 300A, 300B, 300C. The system determines intervention response 304A using data from dataset collection 300A, intervention response 304B using data from dataset collection 300B, and intervention response 304C using data from dataset collection 300C.

As an illustrative example, the datasets 300 associated with the subject may be oncological clinical visit note documents with text authored by clinician(s) (e.g., physician(s)). Each of dataset collections 300A, 300B, 300C may be a set of the three earliest oncological clinical visit note documents generated after respective time points 306A, 306B, 306C. For each collection of oncological clinical visit note documents, the system generates a set of features using text from the oncological clinical visit note documents. The system provides the set of features as input to the response prediction model 302 to obtain a predicted intervention response.

FIG. 4A is a diagram of a machine learning model architecture 400 for extraction of intervention responses from data associated with a subject, according to some embodiments of the technology described herein. In some embodiments, the machine learning model architecture 400 of FIG. 4A may be used by the intervention response extraction system 100 described herein with reference to FIGS. 1A-1D.

As shown in FIG. 4A, the machine learning model architecture 400 includes the time point prediction model 204 described herein with reference to FIGS. 2A-2B and the response prediction model 302 described herein with reference to FIG. 3. The time point prediction model 204 includes parameters (e.g., neural network weights) trained to process sets of features generated from data partitions (e.g., partitions 202A, 202B, 202C) to output time points (e.g., time points 206A, 206B, 206C). The response prediction model 302 includes parameters trained to process sets of features generated from dataset collections (e.g., dataset collections 300A, 300B, 300C) identified based on time points output by the time point prediction model 204 to output predicted intervention responses (e.g., intervention responses 304A, 304B, 304C).

In some embodiments, each of the time point prediction model 204 and the response prediction model 302 may be trained individually (e.g., as described herein with reference to FIG. 2A and FIG. 3). The trained models 204, 302 may then be combined to form the machine learning model architecture 400 of FIG. 4A. In some embodiments, the time point prediction model 204 and the response prediction model 302 may be trained in conjunction. For example, the models 204, 302 may be trained using training data comprising multiple training samples. Each training sample may include: (1) a first set of features generated from a data partition including imaging related data (e.g., radiology report documents) and, optionally, other data (e.g., clinical visit note documents) generated in a respective time period; (2) a time point label corresponding to the first set of features indicating a target time point to be predicted by the time point prediction model 204; (3) a second set of features generated from a collection of datasets (e.g., clinical visit note documents) generated after the time point label; and (4) an intervention response label corresponding to the second set of features indicating a target intervention response to be predicted by the response prediction model 302. The parameters of the models 204, 302 may be trained by applying a supervised learning technique (e.g., stochastic gradient descent) to the training data. The training data samples may be used to determine predicted outputs of the models 204, 302 for input sets of features, compare the predicted outputs of the models 204, 302 to their corresponding labels, and update parameters of the models 204, 302 based on a difference between the predicted outputs and the labels.

The trained parameters of the time point prediction model 204 and the parameters of the response prediction model 302 may be stored in memory for use in intervention response extraction. For example, the parameters of the models 204, 302 may be stored in datastore 108 of the intervention extraction response system 100 described herein with reference to FIGS. 1A-1D. The trained parameters may be used to process data (e.g., clinical record data comprising radiology report documents and clinical visit note documents) associated with a subject to extract intervention responses of the subject.

FIG. 4B is a diagram of another machine learning model architecture 410 for extraction of intervention responses from data associated with a subject, according to some embodiments of the technology described herein. The machine learning model architecture 410 includes the time point prediction model 204 described herein with reference to FIGS. 2A-2B and the response prediction model 302 described herein with reference to FIG. 3. The time point prediction model 204 includes parameters (e.g., neural network weights) trained to process sets of features generated from datasets 212 to output time points time points 216A, 216B, 216C, 216D, 216E. As shown in FIG. 4B, the time point 216C is filtered out from the set of time points used to identify dataset collections from which to determine intervention responses (e.g., because the time point 216C is less than a threshold amount of time after time point 216B). As such, time points 216A, 216B, 216D, 216E are used to identify corresponding dataset collections 310A, 310B, 310D, 310E and time point 216C is not used to identify a corresponding dataset collection. The response prediction model 302 includes parameters trained to process sets of features generated from dataset collections 310A, 310B, 310D, 310E to output predicted intervention responses 314A, 314B, 314D, 314E.

FIG. 5A is an example process 500 of extracting intervention responses for a subject from data associated with the subject, according to some embodiments of the technology described herein. In some embodiments, process 500 may be performed by intervention extraction system 100 described herein with reference to FIGS. 1A-1C.

Process 500 begins at block 502, where the system accesses imaging related data (e.g., radiology report documents) associated with a subject. In some embodiments, the system may access the imaging related data associated with the subject from a database (e.g., datastore 120 described herein with reference to FIG. 1A) storing data associated with subjects. The database may be a corpus of compiled subject data. For example, the system may access the imaging related data associated with the subject from a database storing clinical data associated with subjects obtained from an EHR database.

In some embodiments, the system may access imaging related data generated over a particular time period. For example, the system may access imaging related data generated in a time period following diagnosis of a condition (e.g., diagnosis of a tumor in the subject). As another example, the system may access imaging related data generated in a time period (e.g., a range of dates) specified by a user. In some embodiments, the system may identify imaging related data generated in a given time period based on a timestamp associated with the imaging related data. For example, the system may identify radiology report document(s) generated in a given time period by determining whether timestamps associated with the radiology report document(s) are within the time period.

Next, process 500 proceeds to block 504, where the system divides the imaging related data accessed at block 502 into multiple partitions. In some embodiments, the system may divide the imaging related data into partitions associated with respective time periods. For example, each partition may store imaging related data generated in a particular time period of at least two weeks. In some embodiments, the partitions may represent a sequence of consecutive time periods. As an illustrative example, the system may divide a set of radiology report documents into multiple partitions by: (1) sorting the radiology report documents by generation date (e.g., indicated by timestamps); and (2) dividing the sorted radiology report documents into the multiple partitions, which each partition is associated with a respective time period. Each data partition may include radiology report document(s) generated in a time period associated with the data partition. Example data partitioning techniques that may be performed are described herein with reference to the data partitioning module 102 of FIGS. 1A-IC.

In some embodiments, the system may include data in addition to imaging related data in a given data partition. The system may include, in a data partition, clinical visit note documents generated during a time period associated with the data partition. The additional data may further be used in determining a time point. For example, the system may extract text from clinical visit note documents generated in a time period associated with a data partition, and include the extracted text in the data partition.

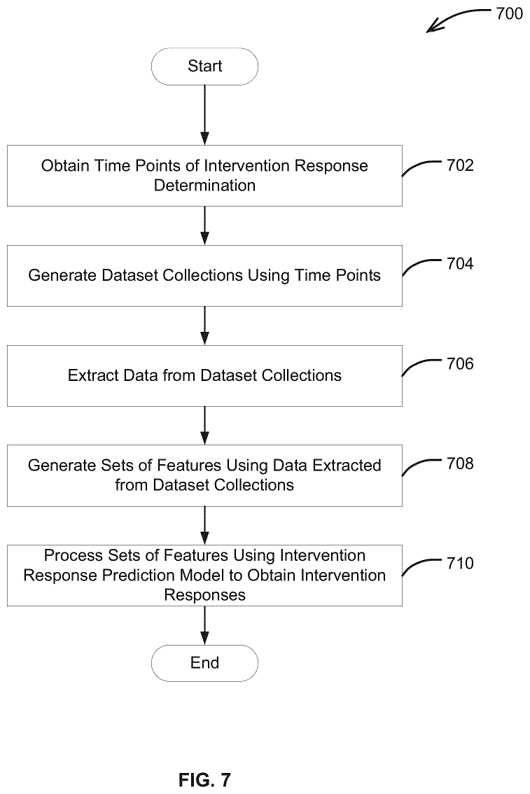

Next, process 500 proceeds to block 506, where the system uses data from the data partitions to determine time points (e.g., dates) indicating predicted time periods of intervention response determinations (e.g., performed by a physician). The system may determine a time point for each of the data partitions. In some embodiments, the system may determine a time point using a data partition by: (1) using data from the data partition to generate a set of features; and (2) processing the set of features using a machine learning model to obtain a time point. An example process for determining a time point using a data partition is described herein with reference to FIG. 5B. FIG. 2A illustrates an example use of data from data partitions to determine time points indicating predicted time periods of intervention response determinations. In some embodiments, the time points determined at block 506 may be used to extract intervention responses from data associated with the subject (e.g., by performing process 700 described herein with reference to FIG. 7).

FIG. 5B is an example process 550 of using data from a data partition storing imaging related data to determine a time point, according to some embodiments of the technology described herein. In some embodiments, process 550 may be performed by intervention response extraction system 100 described herein with reference to FIGS. 1A-1C. In some embodiments, process 550 may be performed at block 506 of process 500 described herein with reference to FIG. 5A.

Process 550 begins at block 552, where the system extracts data (e.g., text) from a data partition. In some embodiments, the system may extract data from the data partition by extracting text snippets from the imaging related data and/or other data of the partition. For example, the system may extract text snippets from radiology report document(s) and/or clinical visit note document(s) included in the data partition. In some embodiments, the system may extract text snippets from the data in the partition by: (1) searching for one or more keywords in data (e.g., document(s)) of the partition; and (2) extracting snippet(s) of text surrounding identified keyword(s) in the data. For example, the system may extract a snippet of text within a certain number of characters or words following and/or preceding an identified keyword. In some embodiments, the system may extract data from the partition by extracting values of one or more database fields. For example, the system may access values of metadata fields for documents included in the data partition.

Next, process 550 proceeds to block 554, where the system generates a set of features using the data extracted from the data partition. In some embodiments, the system may generate an embedding of data extracted from the data partition. The embedding may comprise number values (e.g., real number values) representing the extracted data (e.g., extracted text). The embedding may be one or more vectors or matrices of real number values representing the extracted data. For example, the extracted text may include a text snippet. The system may tokenize the text snippet to obtain a token vector. For example, each value in the token vector may be one or more words from the text snippet. The system may generate an embedding for each token of the token vector (e.g., by determining a vector of 128 real number values representing the token). The system may combine the embeddings generated for the tokens as a set of features representing the extracted text. As another example, the extracted data may include a value of a field (e.g., a metadata field). The system may generate an embedding representing the field value. The system may combine the embedding representing the field value with one or more embeddings representing extracted text to obtain the set of features.

Next, process 550 proceeds to block 556, where the system processes the set of features using a trained machine learning model to obtain a time point indicating a predicted time period in which an intervention response was determined for the subject. For example, the trained machine learning model may be time point prediction model 204 described herein with reference to FIGS. 2A-2B. The system may process the set of features using parameters of the time point prediction model (e.g., accessed from memory of the system). For example, the trained machine learning model be a neural network model and the set of features may be provided as input to an input layer of the neural network model. The system may use parameters (e.g., weights) of layers of the model to determine an output. In some embodiments, the trained machine learning model may output a classification result. The classification result may be values (e.g., a probability values) each associated with a respective one of multiple possible time points (e.g., dates in a year). The system may select a time point with the highest value to be the time point indicative of the intervention response determination for the subject.

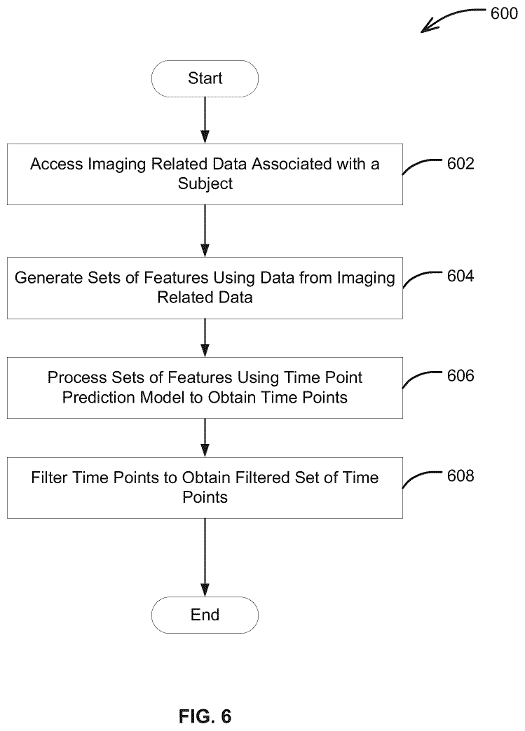

FIG. 6 is another example process 600 of identifying dataset collections from which to extract intervention responses, according to some embodiments of the technology described herein. In some embodiments, process 600 may be performed by intervention response extraction system 100 described herein with reference to FIGS. 1A and 1D.

Process 600 begins at block 602, where the system accesses imaging related data associated with a subject. The system may access imaging related data as described at block 502 described herein with reference to FIG. 5B.

Next, process 600 proceeds to blocks 604-606 where the system processes the imaging related data using a trained machine learning model to obtain time points indicating respective predicted time periods of intervention response determination for a subject. At block 604, the system generates sets of features using data from the imaging related data. In some embodiments, the system may generate the sets of features by extracting text from the imaging related data and using the extracted text to generate the sets of features. The system may generate numeric representations of the text (e.g., numeric vectors) that can be provided as input to the trained machine learning model.

In some embodiments, the system may generate a set of features corresponding to each dataset (e.g., imaging report document) in the imaging related data. The system may generate an embedding of text from a dataset. The embedding may comprise numeric values (e.g., real number values) representing the text. The embedding may be one or more vectors or matrices of real number values representing the extracted text. For example, the extracted text may include a text snippet. The system may tokenize the text snippet to obtain a token vector where each value in the token vector may be one or more words from the text snippet. The system may generate an embedding for each token of the token vector (e.g., by determining a vector of 128 real number values representing the token). The system may combine the embeddings generated for the tokens as a set of features representing text extracted from the dataset. As another example, the extracted data may include a value of a field (e.g., a metadata field). The system may generate an embedding representing the field value. The system may combine the embedding representing the field value with one or more embeddings representing extracted text to obtain the set of features.

Next, at block 606, the system processes the sets of features using a trained machine learning model to obtain time points. The time points may indicate predicted time periods in which an intervention response was determined for the subject. For example, the trained machine learning model may be time point prediction model 204 described herein with reference to FIGS. 2A-2B. The system may process the sets of features using parameters of the time point prediction model (e.g., accessed from memory of the system). For example, the trained machine learning model may be a neural network model and the sets of features may be provided as input to an input layer of the neural network model. The system may use parameters (e.g., weights) of layers of the model to determine outputs. In some embodiments, the trained machine learning model may output classification results for each of the sets of features. The classification results may each include values (e.g., a probability values) associated with respective multiple possible time points (e.g., dates in a year). The system may select a time point with the highest value to be the time point determined for a respective set of features.

Next, process 600 proceeds to block 608, where the system filters the time points obtained at block 606 to obtain a subset of time points. In some embodiments, the system may filter the time points by generating a filtered set of time points in which each pair of time points is separated by at least a threshold amount of time (e.g., two weeks). The system may filter the time points by: (1) identifying any time points that are less than the threshold amount of time after a preceding time point; and (2) remove any identified time points to obtain the filtered set of time points. In some cases, the filtered set of time points may be a subset of the time points (e.g., because one or more of the time points were less than the threshold amount of time after respective preceding time point(s)). In some cases, the filtered set of time points may include all the time points obtained at block 606 (e.g., because all pairs of time points were separated by at least the threshold amount of time). In some embodiments, the filtered set of time points may be used to extract intervention responses from data (e.g., clinical record data) associated with the subject (e.g., by performing process 700 described herein with reference to FIG. 7).

FIG. 7 is an example process 700 of determining an intervention response for a subject using data associated with the subject, according to some embodiments of the technology described herein. In some embodiments, process 700 may be performed by intervention response extraction system 100 described herein with reference to FIGS. 1A-1D.

Process 700 begins at block 702, where the system obtains time points indicating a predicted time periods in which an intervention response was determined for the subject. For example, the time points may be obtained by performing processes 500, 550 described herein with reference to FIG. 5B or process 600 described herein with reference to FIG. 6.

Next, process 700 proceeds to block 704, where the system generates dataset collections using the time points. Each of the dataset collections may include one or more. For example, the system may identify a collection of clinical visit note document(s) using the time point. In some embodiments, the system may generate a dataset collection corresponding to each time point. The system may generate a dataset collection corresponding to a time point by: (1) identifying one or more datasets (e.g., clinical note documents) generated after the time point; and (2) store the identified dataset(s) in the dataset collection. In some embodiments, the system may identify a particular number (e.g., three) of earliest generated datasets after the time point. For example, the system may identify the three earliest oncology clinical visit note documents generated after the time point to use in the determination of an intervention response. In some embodiments, the system may identify dataset(s) generated within a threshold amount of time (e.g., 1 week, 2 weeks, 3 weeks, or another suitable amount of time) after the time point.

Next, process 700 proceeds to blocks 706-710 where the system processes the dataset collections using a trained machine learning model to obtain intervention responses. At block 706, the system extracts data from the dataset collections. In some embodiments, the system may extract data from a dataset collection by extracting text snippets from dataset(s) in the collection. For example, the system may extract text snippets from clinical visit note document(s) included in the collection. In some embodiments, the system may extract text snippets from the dataset collection by: (1) searching for one or more keywords in datasets (e.g., document(s)) of the collection; and (2) extracting snippet(s) of text surrounding identified keyword(s) in the data. For example, the system may extract a snippet of text within a certain number of characters or words following and/or preceding an identified keyword. In some embodiments, the system may extract data from the dataset collection by extracting values of one or more fields. For example, the system may access values of metadata fields for clinical visit note documents included in the data partition.

Next, process 700 proceeds to block 708, where the system generates sets of features using the data extracted from the dataset collection. In some embodiments, the system may generate embeddings of number values (e.g., real number values) representing the extracted data. The embeddings may each be one or more vectors or matrices of real number values representing data extracted from a respective data collection. For example, extracted data may include a text snippet. The system may tokenize the text snippet to obtain a token vector. For example, each value in the token vector may be one or more words from the text snippet. The system may generate an embedding for each token of the token vector (e.g., by determining a vector of 128 real number values representing the token). The system may combine the embeddings generated for the tokens as a set of features representing the extracted text. As another example, the extracted data may include a value of a field (e.g., a metadata field). The system may generate an embedding representing the field value. The system may combine the embedding representing the field value may with one or more embeddings representing extracted text to obtain the set of features.

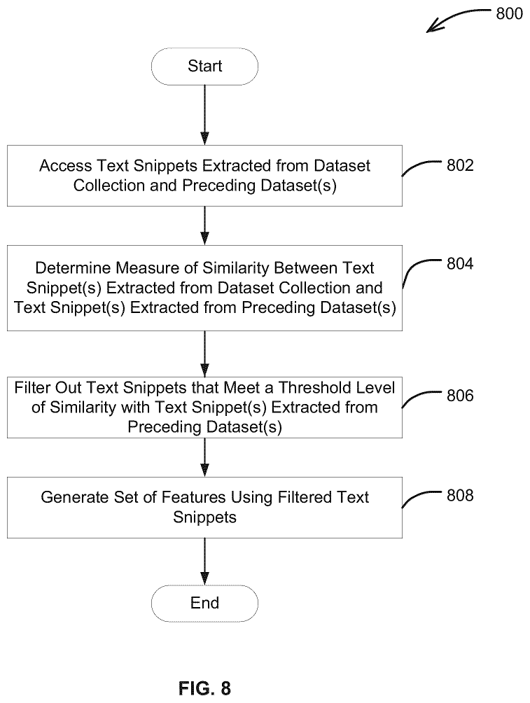

In some embodiments, the system may apply filtering to the extracted set of data as part of generating the set of features. The system may filter data that is similar or identical to data in a previously processed dataset collection. For example, the system may filter out text snippets that are identical or sufficiently similar to text snippets extracted from a previously processed dataset collection. An example process of filtering the data is described herein with reference to FIG. 8.

In some embodiments, the system may generate a set of features using data from a dataset collection by determining a previously determined intervention response and including an indication of the previously determined intervention response in the set of features. For example, the system may determine an intervention response determined (e.g., by performing process 700) based on a time point immediately preceding the time point obtained at block 702. The system may include an indication of the intervention response in the set of features. For example, the previously determined intervention response may be a classification into one of multiple different response categories, and the system may include a numerical value associated with the response category of the classification in the set of features. To illustrate, the previously determined intervention response may be a classification of complete response (CR) associated with a value of 1. The system may include a value of 1 as a feature in the set of features.

After the generation of the set of features at block 708, process 700 proceeds to block 710, where the system processes the sets of features using an intervention response prediction model to obtain intervention responses. For example, the intervention response prediction model may be the intervention response prediction model 302 described herein with reference to FIG. 3. The system may process the sets of features using parameters of the intervention response prediction model (e.g., accessed from memory of the system). For example, the intervention response prediction model may be a neural network model and the set of features may be provided as input to an input layer of the neural network model. The system may use parameters (e.g., weights) of layers of the model to determine an output for a set of features. In some embodiments, the intervention response prediction model may output a classification result. The classification result may be a value (e.g., a probability value) associated with each of multiple possible intervention response categories (e.g., CR, PR, CD, SD, and unknown). The system may select a response category with the highest value to be the predicted intervention response.

FIG. 8 is an example process 800 of filtering data extracted from a dataset collection (e.g., a collection of clinical documents), according to some embodiments of the technology described herein. In some embodiments, process 800 may be performed by intervention response extraction system 100 described herein with reference to FIGS. 1A-1D. In some embodiments, process 800 may be performed at block 708 of process 700 described herein with reference to FIG. 7.

Process 800 begins at block 802, where the system accesses text snippets extracted from a dataset collection currently being processed and one or more preceding datasets (e.g., the last 1, 2, 3, 4, 5, 6, 7, 8, 9, or 10 datasets prior to the dataset collection). Text snippets may have been extracted from datasets as described herein at block 706 of process 700.

Next, process 800 proceeds to block 804, where the system determines a measure of similarity between text snippet(s) extracted from the dataset collection and the text snippet(s) extracted from the preceding dataset(s). In some embodiments, the system may determine a measure of similarity between a pair of text snippets by determining a degree of overlap between the two text snippets. For example, the system may determine a percentage of overlap between the two text snippets. The system may determine a measure of similarity between the text snippet(s) extracted from the dataset collection and each text snippet extracted from the preceding dataset(s).

Next, process 800 proceeds to block 806, where the system filters out text snippets that meet a threshold level of similarity with a text snippet extracted from the preceding dataset(s). In some embodiments, the system may filter out text snippets that have a minimum degree of overlap with a text snippet extracted from the preceding dataset(s). For example, the system may filter out text snippets that have at least an 80% overlap with a text snippet extracted from the preceding dataset(s). The system may thus eliminate data related to an intervention response associated with the preceding dataset(s) in extracting an intervention response from the current dataset collection.

Next, process 800 proceeds to block 808, where the system uses the filtered text snippets to generate a set of features. The system may generate a set of features as described at block 706 of process 700.

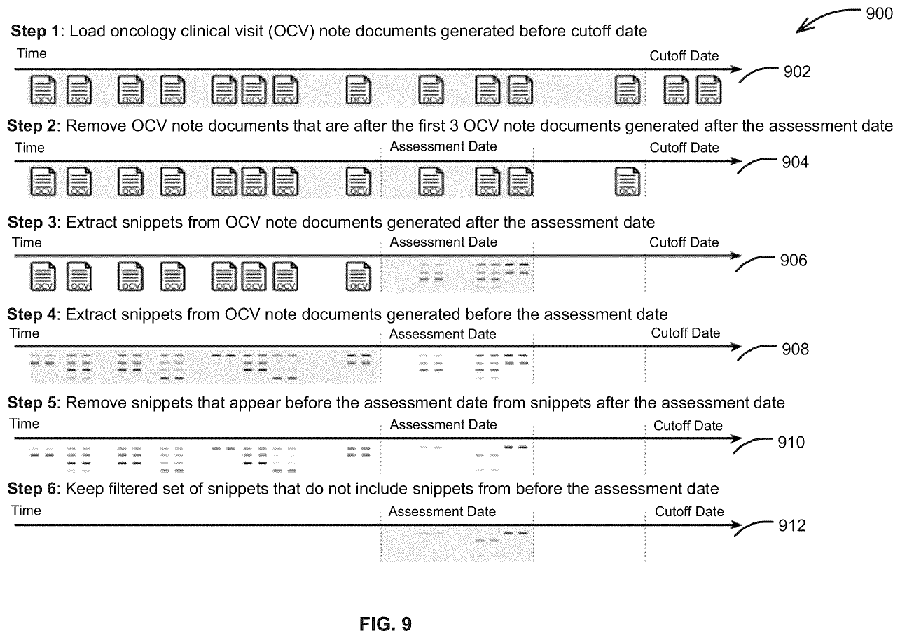

FIG. 9 is a diagram 900 depicting filtering of data for a collection of datasets, according to some embodiments of the technology described herein. The filtering depicted by the diagram 900 may be performed as part of process 800 described herein with reference to FIG. 8 (e.g., by intervention response extraction system 100 described herein with reference to FIGS. 1A-1C). In the example of FIG. 9, filtering is being performed for a collection OCV note documents determined based on a time point (referred to as “assessment date”). Although the example of FIG. 9 filters OCV note documents, some embodiments may be configured to perform filtering for other types of datasets in addition to or instead of OCV note documents.

In the first step 902 of the filtering, OCV note documents prior to a cutoff date are loaded by the system. In the second step 904, the system identifies a collection of OCV note documents generated based an assessment date. For example, the assessment date may be a timepoint identified by performing process 600 described herein with reference to FIG. 5B. In the example of FIG. 9, the system identifies a collection of the three earliest OCV note documents generated after the assessment date. In the second step 904, the system removes OCV note documents generated after the three earliest OCV note documents.

In the third step 906, the system extracts text snippets from the collection of OCV note documents. In the fourth step 908, the system extracts text snippets from OCV note documents preceding the collection of OCV note documents (i.e., before the assessment date). In the fifth step 910, the system filters out text snippets in the collection of OCV note documents that appeared in the preceding OCV note documents (e.g., by determining that the text snippets have a threshold similarity to text snippets that appeared in the preceding OCV note documents) to obtain a filtered set of text snippets extracted from the collection of OCT note documents. In the sixth step 912, the system keeps the filtered set of text snippets extracted from the collection of OCV note documents (e.g., for use in generating a set of features to determine an intervention response).

By performing the filtering depicted in FIG. 9, the system may eliminate text in the collection of OCV notes that is repeated from previous OCV note documents. This may allow the system to use text from the collection of OCV notes that is new (e.g., generated after the assessment date) relative to the previous OCV note documents. The system may thus ensure that the system is not using previously authored text that may not relate to an intervention response determined after the assessment date.

FIG. 10 shows a block diagram of an example computer system 1000 that may be used to implement some embodiments of the technology described herein. The computing device 1000 may include one or more computer hardware processors 1002 and non-transitory computer-readable storage media (e.g., memory 1004 and one or more non-volatile storage devices 1006). The processor(s) 1002 may control writing data to and reading data from (1) the memory 1004; and (2) the non-volatile storage device(s) 1006. To perform any of the functionality described herein, the processor(s) 1002 may execute one or more processor-executable instructions stored in one or more non-transitory computer-readable storage media (e.g., the memory 1004), which may serve as non-transitory computer-readable storage media storing processor-executable instructions for execution by the processor(s) 1002.

Having thus described several aspects of at least one embodiment of the technology described herein, it is to be appreciated that various alterations, modifications, and improvements will readily occur to those skilled in the art.

Such alterations, modifications, and improvements are intended to be part of this disclosure, and are intended to be within the spirit and scope of disclosure. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semicustom or custom. As a specific example, some commercially available microprocessors have multiple cores such that one or a subset of those cores may constitute a processor. However, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the technology as described above. As used herein, the term “computer-readable storage medium” encompasses only a non-transitory computer-readable medium that can be considered to be a manufacture (i.e., article of manufacture) or a machine. Alternatively or additionally, aspects of the technology described herein may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the technology as described above. Additionally, it should be appreciated that according to one aspect of this embodiment, one or more computer programs that when executed perform methods of the technology described herein need not reside on a single computer or processor, but may be distributed in a modular fashion amongst a number of different computers or processors to implement various aspects of the technology described herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the technology described herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the technology described herein may be embodied as a method, of which examples are provided herein including with reference to FIGS. 3 and 7. The acts performed as part of any of the methods may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by an “actor” or a “user”. It should be appreciated that an “actor” or a “user” need not be a single individual, and that in some embodiments, actions attributable to an “actor” or a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A computer system for automatically extracting intervention responses from data associated with subjects, the computer system comprising:
at least one processor; and
a plurality of modules executed by the at least one processor, the plurality of modules comprising a time point extraction module, a response data identification module, and a response extraction module, wherein:
the time point extraction module is configured to:
access imaging related data associated with a subject;
process the imaging related data using a first trained machine learning (ML) model to obtain a plurality of time points, the processing comprising:
generate a plurality of sets of features using data from the image related data; and
process the plurality of sets of features using the first trained ML model to obtain the plurality of time points;
the response data identification module is configured to:
generate, using the data associated with the subject, a dataset collection for each of at least some of the plurality of time points to obtain a plurality of dataset collections, the generating comprising:
identify, in the data associated with the subject, one or more datasets generated after the time point; and
include the one or more datasets in the dataset collection; and
the response extraction module is configured to:
process the plurality of dataset collections using a second trained ML model to obtain a plurality of intervention responses, the processing comprising, for each of at least some of the plurality of dataset collections:
generate a set of features using data from the dataset collection; and
process the set of features using the second trained ML model to obtain an intervention response of the plurality of intervention responses.

2. The system of claim 1, wherein the first trained ML model is a first neural network model, and the second trained ML model is a second neural network model.

3. The system of claim 1, wherein the response extraction module is further configured to generate a dataset storing the at least some time points and corresponding intervention responses obtained using dataset collections generated using the at least some time points.

4. The system of claim 3, wherein generating a dataset storing the at least some time points and corresponding intervention responses obtained using dataset collections generated using the at least some time points comprises storing, in the dataset:
a first time point of the at least some time points;
a first intervention response corresponding to the first time point, the first intervention response obtained using data from a first one of the plurality of dataset collections, the first dataset collection comprising one or more datasets generated after the first time point;
a second time point of the at least some time points, the second time point subsequent to the first time point; and
a second intervention response corresponding to the second time point, the second intervention response obtained using data from a second one of the plurality of dataset collections, the second dataset collection comprising one or more datasets generated after the second time point.

5. The system of claim 1, wherein the plurality of time points comprises a plurality of dates.

6. The system of claim 1, wherein each of at least some of the plurality of intervention responses is one of a group consisting of: complete response, partial response, stable disease, progressive disease, and unknown.

7. The system of claim 1, wherein the at least some time points are a subset of the plurality of time points and the time point extraction module is further configured to:
identify the subset of time points such that each pair of the subset of time points is separated by at least a threshold amount of time.

8. The system of claim 7, wherein identifying the subset of time points comprises:
identifying one or more of the plurality of time points that are less than the threshold amount of time after a respective preceding time point of the plurality of time points; and
filtering out the one or more time points from the plurality of time points to obtain the subset of time points.

9. The system of claim 7, wherein the threshold amount of time is two weeks.

10. The system of claim 1, wherein the plurality of modules further comprises a data partitioning module configured to:
divide the imaging related data into a plurality of partitions;
wherein generating the plurality of sets of features using the data from the image related data comprises using each of the plurality of partitions to generate a respective one of the plurality of sets of features.

11. The system of claim 10, wherein:
the imaging related data comprises a plurality of imaging report documents;
the plurality of partitions are associated with a respective plurality of time periods; and
dividing the imaging related data into the plurality of partitions comprises:
dividing the plurality of imaging report documents into the plurality of partitions by storing, in each of the plurality of partitions, one or more of the plurality of imaging report documents generated in a respective time period associated with the partition.

12. The system of claim 1, wherein identifying, in the data associated with the subject, one or more datasets generated after the time point comprises:
identifying one or more earliest generated datasets after the time point.

13. The system of claim 12, wherein identifying the one or more earliest generated datasets generated after the time point comprises identifying three earliest generated datasets after the time point.

14. The system of claim 1, wherein the at least one processor is further configured to obtain the first trained ML model by performing training using training data comprising:
sets of features generated using imaging related data associated with a plurality of subjects; and
time point labels indicating target time point predictions for the sets of features.

15. The system of claim 1, wherein the at least one processor is configured to obtain the second trained ML model by performing training using training data comprising:
sets of features generated from datasets associated with a plurality of subjects; and
intervention response labels indicating target intervention response predictions for the sets of features.

16. The system of claim 1, wherein generating the plurality of sets of features using data from the image related data comprises generating the plurality of sets of features using text extracted from the image related data.

17. The system of claim 1, wherein generating the set of features using data from the dataset collection comprises:
extracting text from the dataset collection; and
generating the set of features using the text extracted from the dataset collection.

18. The system of claim 17, wherein the text extracted from the dataset collection includes a first set of text and the response extraction module is further configured to:
extract a second set of text from one or more datasets generated before one or more datasets of the dataset collection;
determine a measure of similarity between the first set of text and the second set of text;
determine that the measure of similarity meets a threshold level of similarity; and
remove the first set of text from the text extracted from the dataset collection when the measure of similarity meets the threshold similarity to obtain a filtered set of text;
wherein generating the set of features using the text extracted from the dataset collection comprises generating the set of features using the filtered set of text.

19. A method for automatically extracting intervention responses from data associated with subjects, the method comprising:
using at least one processor to perform:
accessing imaging related data associated with a subject;
processing the imaging related data using a first trained machine learning (ML) model to obtain a plurality of time points, the processing comprising:
generating a plurality of sets of features using data from the image related data;
processing the plurality of sets of features using the first trained ML model to obtain the plurality of time points;
identifying, from the data associated with the subject, a first collection of one or more datasets generated after a first one of the plurality of time points;
generating, using the data associated with the subject, a dataset collection for each of at least some of the plurality of time points to obtain a plurality of dataset collections, the generating comprising:
identifying, in the data associated with the subject, one or more datasets generated after the time point; and
including the one or more datasets in the dataset collection; and
processing the plurality of dataset collections using a second trained ML model to obtain a plurality of intervention responses, the processing comprising, for each of at least some of the plurality of dataset collections:
generating a set of features using data from the dataset collection; and
processing the set of features using the second trained ML model to obtain an intervention response of the plurality of intervention responses.

20. A non-transitory computer-readable medium storing instructions that, when executed by at least one processor, cause the at least one processor to perform a method for automatically extracting intervention responses from data associated with subjects, the method comprising:
accessing imaging related data associated with a subject;
processing the imaging related data using a first trained machine learning (ML) model to obtain a plurality of time points, the processing comprising:
generating a plurality of sets of features using data from the image related data;
processing the plurality of sets of features using the first trained ML model to obtain the plurality of time points;
identifying, from the data associated with the subject, a first collection of one or more datasets generated after a first one of the plurality of time points;
generating, using the data associated with the subject, a dataset collection for each of at least some of the plurality of time points to obtain a plurality of dataset collections, the generating comprising:
identifying, in the data associated with the subject, one or more datasets generated after the time point; and
including the one or more datasets in the dataset collection; and
processing the plurality of dataset collections using a second trained ML model to obtain a plurality of intervention responses, the processing comprising, for each of at least some of the plurality of dataset collections:
generating a set of features using data from the dataset collection; and
processing the set of features using the second trained ML model to obtain an intervention response of the plurality of intervention responses.
